(19) United States
(12) Patent Application Publication
(10) Pub. No.: US 2025/0326830 A1
(43) Pub. Date: Oct. 23, 2025

(54) TREATMENT OF OPHTHALMOLOGIC DISEASES

(71) Applicant: Hoffmann-La Roche Inc., Little Falls, NJ (US)

(72) Inventors: Andreas MAUNZ, Freiburg (DE); Sascha FAUSER, Basel (CH); Carl-Gustav OLSEN GLITTENBERG, Arlesheim (CH)

(73) Assignee: Hoffmann-La Roche Inc., Little Falls, NJ (US)

(21) Appl. No.: 19/182,917

(22) Filed: Apr. 18, 2025

(30) Foreign Application Priority Data
Apr. 19, 2024 (WO) PCT/US2024/025311

(51) Int. Cl. C07K 16/22; A61K 39/00; A61P 27/02

(52) U.S. Cl. CPC C07K 16/22; A61P 27/02; A61K 2039/505; A61K 2039/545

(57) ABSTRACT

The current invention relates to antibodies, which bind to VEGF and ANG2 for use in the treatment of ocular vascular diseases.

### CROSS-REFERENCE TO RELATED APPLICATIONS

This application claims priority to International Patent Application no. PCT/US2024/025311, filed Apr. 19, 2024, which is incorporated by reference in its entirety

### SEQUENCE LISTING

This application contains a Sequence Listing which has been submitted electronically in XML format and is hereby incorporated by reference in its entirety. Said XML copy, created on Apr. 14, 2025, is named P39310-US_SEQ_List.xml and is 25,386 bytes in size.

### FIELD OF THE DISCLOSURE

The current invention relates to antibodies that bind to VEGF and ANG2 for use in the treatment of ocular vascular diseases

### BACKGROUND OF THE DISCLOSURE

Ocular vascular diseases such as diabetic retinopathy in particular diabetic macular edema (DME) are severe diseases leading to often to visual loss and blindness. DME, a complication of diabetic retinopathy (DR), can develop at any stage of the underlying disease of retinal microvasculature. DME occurs with increasing frequency as the underlying DR worsens from non-proliferative DR (NPDR) to proliferative DR (PDR). DME is the most common cause of moderate and severe visual impairment in patients with DR, and if left untreated can lead to a loss of 10 or more letters in visual acuity (VA) within 2 years in approximately 50% of patients. DME affects approximately 14% of patients with diabetes and can be found in patients with both Type 1 and Type 2 diabetes. In 2013, the worldwide population of people with diabetes was approximately 382 million, and it is estimated to grow to 592 million by 2035 (International Diabetes Federation 2013).

With advances in imaging technology, DME is now often diagnosed by optical coherence tomography (OCT) rather than the traditional Early Treatment Diabetic Retinopathy Study (ETDRS) ophthalmoscopy-based criteria. On a molecular level, DME is a result of a vascular endothelial growth factor-A (VEGF-A)-mediated increase in vessel permeability and loss of pericytes, consequent to hypoxia-mediated release of pro-angiogenic, hyperpermeability, and pro-inflammatory mediators. VEGF also upregulates a homeostatic factor, angiopoietin-2 (Ang-2), which acts as an antagonist of the Tie2 receptor tyrosine kinase on endothelial cells, counteracting vessel stabilization maintained through Ang-1-dependent Tie2 activation. Therefore, Ang-2 acts as a vascular destabilization factor, rendering the vasculature more elastic and amenable to endothelial barrier breakdown and sprouting. The excess of Ang-2 and VEGF in the retinal tissues promotes vessel destabilization, vascular leakage, and neovascularization. Ang-2 is also involved in inflammatory pathways such as lymphocyte recruitment. In summary, both VEGF-A and Ang-2 are recognized as key factors mediating diabetic eye disease pathogenesis.

Although macular laser used to be the standard of care (SOC) for treatment of DME, the development of anti-VEGF pharmacotherapy in the past 10 years has led to dramatic improvements in visual outcomes for patients with DME. Other available approved options for the treatment of DME include periocular or intravitreal (IVT) steroids and steroid implants.

Despite the strong efficacy achieved with anti-VEGF therapies in DME, a significant proportion of patients do not experience clinically meaningful improvements in vision in the real world. Frequent IVT administration is required to achieve, and in some cases, to maintain the observed early benefits of DME treatment over a long period of time. The current SOC for administration of anti-VEGF injections requires patients to undergo frequent clinical examinations and IVT injections. This imposes a significant burden on patients, caregivers, treating physicians, and the healthcare system.

In order to better address the complex nature of DME and improve long-term outcomes, attempts have been made to target more than one pathway involved in its pathogenesis. Faricimab (Vabysmo, F. Hoffmann-La Roche), a bispecific antibody that blocks both Angiopoietin-2 (Ang-2) and VEGF-A, is one of these molecules.

The Ang/Tie pathway is a key player in the development and homeostasis of vessels. Activation of Tie2 by Ang-1 leads to vascular stability. Ang-2 on the other hand acts predominantly as an antagonist of Ang-1. When Ang-2 is upregulated, as is the case in multiple retinal pathologies including diabetic retinopathy, it destabilizes the vasculature and enhances the vessels' sensitivity to VEGF-A. Preclinical studies have shown that Ang-2 and VEGF-A act in synergy to drive vascular leakage, neovascularization and inflammation, making combined inhibition of Ang-2 and VEGF-A a potentially valuable approach to improve vascular stability, and as a result disease severity and long-term outcomes.

Hyperreflective foci (HRF) have been proposed as a biomarker of disease severity and progression in DME. HRF are small, distinct objects that generate a highly reflective signal on spectral-domain optical coherence tomography (SD-OCT). They are present in multiple retinal diseases, including DME, neovascular age-related macular degeneration, retinal vein occlusion, and uveitic macular edema. In patients receiving treatment for DME, the presence or number of HRF at baseline has been shown to be predictive of a poor visual outcome despite treatment. Treatment with intravitreal anti-VEGF or steroids reduces the number of HRF, with several studies indicating that steroids have a greater effect, suggesting that DME patients with high HRF burden benefit from an additional mode of action beyond anti-VEGF. Therefore, there remains a need for improving HFR burden and improving severity and progression in DME.

### SUMMARY OF THE DISCLOSURE

One aspect of the disclosure provides a method of reducing hyperreflective foci (HRF) in an eye of a patient. Such method includes administering to the patient an effective amount of a bispecific antibody which binds to human vascular endothelial growth factor (VEGF) and to human angiopoietin-2 (ANG-2). In certain embodiments, such patient suffers from diabetic macular edema (DME).

Another aspect of the disclosure provides a bispecific antibody which binds to human VEGF and to human ANG-2 for use in method of reducing HRF in an eye of a patient. In certain embodiments, such patient suffers from DME.

Another aspect of the disclosure provides a formulation comprising a bispecific antibody which binds to human VEGF and to human ANG-2 for use in method of reducing HRF in an eye of a patient. In certain embodiments, such patient suffers from DME.

Another aspect of the disclosure provides a method of treating a patient suffering from DME. Such method includes administering to the patient an effective amount of a bispecific antibody which binds to human VEGF and to human ANG-2; and measuring hyperreflective foci (HRF) in an eye of the patient after 16 and/or 48 weeks of treatment.

Yet another aspect of the disclosure provides a method of treating a patient suffering from DME. Such method includes administering to the patient an effective amount of a bispecific antibody which binds to human VEGF and to human ANG-2; measuring hyperreflective foci (HRF) in an eye of the patient after 16 and/or 48 weeks of treatment; and adjusting administration dosing interval based on the HRF volume and/or count.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 is a diagram showing study design overview. *The personalized treatment interval (PTI) is a protocol-driven regimen based on the treat-and-extend concept. †Change from baseline in best-corrected visual acuity (BCVA), as measured on the ETDRS chart at a starting distance of 4 m at 1 year, is the average of the week 48, 52, and 56 visits. Q8W=every 8 weeks; R=randomization.

FIG. 2 illustrates the model trained and validated on phase 2 BOULEVARD volume scans (90% training and 10% holdout/validation set). Subsequently the model was applied to YOSEMITE and RHINE volume scans to quantify HRF.

FIG. 3 is an example of automated HRF segmentation: Example SD-OCT images from one patient (age 67, female, faricimab T&E arm) at baseline without (a) and with segmentation (b) of layers, HRF (red), and larger hyperreflective objects (green). Image from the same patient at week 48 without (c) and with segmentation of HRF (d). ETDRS rings are indicated with green vertical lines (center, 1-mm, and 3-mm diameter ring). Inner retina (between Internal limiting membrane (ILM) and outer plexiform layer-Henle's fiber layer (OPL-HFL) purple, and outer retina (between OPL-HFL and retinal pigment epithelium (RPE) green. CST: central subfield thickness.

FIG. 4 shows volumetric analysis of HRF in the inner and outer retina over time. Inner retina 1 mm (a) and 3-mm (b) diameter, outer retina 1-mm (c) and 3-mm (d) diameter ETDRS rings. Results and nominal p-values were obtained using a MMRM analysis. Since the model is adjusted for baseline HRF value, no baseline values are shown in the figure. CI: confidence interval; MMRM: mixed model for repeated measures.

FIG. 5 shows baseline and week 48 measured HRF volumes in the inner retina 1-mm and 3-mm diameter, and outer retina 1-mm and 3-mm diameter ETDRS rings for faricimab Q8W (black), faricimab T&E (light gray), and aflibercept Q8W (dark grey). Nominal P-values, derived from the MMRM, are indicated as: ** P≤0.01, *** P≤0.001, **** P≤0.0001. Values are square-root transformed for better visibility.

FIG. 6 shows HRF volume as a proportion of baseline at week 48 based on the median (or Q3 if median zero).

FIG. 7 provides a flowchart showing model training, validation, and post-processing for HRF quantification. 2-D: two dimensional; IHRM: intraretinal hyperreflective material.

FIG. 8 shows HRF annotation and volume calculation: An HRF is assumed to be an ellipsoid with the length a (measured along the longest axis of the ellipsoid), which is used as a filter criterion in the post-processing step. The volume of an HRF object is computed as the product of the actual 2-D area and the depth d, which is defined as the distance between B-Scans.

FIG. 9 shows scatterplot of hold-out ground truth vs segmented HRF and IHRM volumes, including a linear best-fit line.

FIG. 10 shows boxplots of Chamfer distances for internal limiting membrane (ILM), outer plexiform layer-Henle's fibre layer (OPL-HFL), and retinal pigment epithelium (RPE). Px: pixels; StdErr: Standard error; IQR: interquartile range; Avg: average; StdDev: standard deviation.

FIG. 11 shows volumetric analysis of HRF in the total retina over time. 1-mm (a) and 3-mm (b) diameter ETDRS rings. Results and nominal p-values were obtained using a MMRM analysis. Since the model is adjusted for baseline HRF value, no baseline values are shown in the figure.

FIG. 12 shows HRF counts in the inner and outer retina over time. Inner retina 1-mm (a) and 3-mm (b) diameter, outer retina 1-mm (c) and 3-mm (d) diameter ETDRS rings. Results and nominal p-values were obtained using a MMRM analysis. Since the model is adjusted for baseline HRF value, no baseline values are shown in the figure.

### DETAILED DESCRIPTION OF THE DISCLOSURE

As provided above, one aspect of the disclosure provides a method of reducing hyperreflective foci (HRF) in an eye of a patient. Such method includes administering to the patient an effective amount of a bispecific antibody which binds to human vascular endothelial growth factor (VEGF) and to human angiopoietin-2 (ANG-2).

In certain embodiments, the patient suffers from DME.

Another aspect of the disclosure provides a bispecific antibody which binds to human VEGF and to human ANG-2 for use in method of reducing HRF in an eye of a patient. In certain embodiments, such patient suffers from DME.

Another aspect of the disclosure provides a formulation comprising a bispecific antibody which binds to human VEGF and to human ANG-2 for use in method of reducing HRF in an eye of a patient. In certain embodiments, such patient suffers from DME.

Another aspect of the disclosure provides a method of treating a patient suffering from DME. Such method includes administering to the patient an effective amount of a bispecific antibody which binds to human VEGF and to human ANG-2; and measuring hyperreflective foci (HRF) in an eye of the patient after 16 and/or 48 weeks of treatment. In certain embodiments, the method further includes adjusting administration dosing interval based on the HRF volume and/or count.

In certain embodiments of the methods or bispecific antibodies (for use), medicaments or pharmaceutical formulations of the disclosure, the effective amount of the bispecific antibody is sufficient to reduce HRF volume and/or count after 16 weeks of treatment or longer. In certain embodiments, the effective amount of the bispecific antibody is sufficient to reduce HRF volume and/or count after 48 weeks of treatment or longer.

In certain embodiments, the HRF volume after 48 weeks of treatment is less than about 0.5 relative to the HRF volume prior to treatment. In other embodiments, the HRF volume after 48 weeks of treatment is less than about 0.45, 0.4, 0.35, or 0.3 relative to the HRF volume prior to treatment.

In certain embodiments, the HRF volume after 48 weeks of treatment is less than about 0.8 relative to the HRF volume after the standard of care treatment (such as aflibercept). In other embodiments, the HRF volume after 48 weeks of treatment is less than about 0.9, 0.85, 0.75, 0.7, 0.65, or 0.6 relative to the HRF volume after the standard of care treatment.

In certain embodiments of the methods or bispecific antibodies (for use), medicaments or pharmaceutical formulations of the disclosure, the HRF volume is reduced in the inner retina and the outer retina. In some embodiments, the HRF volume is reduced in the inner retina. In some embodiments, the HRF volume is reduced in the outer retina.

The HRF volume may be reduced in the central 1-mm diameter of the retina and/or reduced in the central 3-mm diameter of the retina. In certain embodiments of the methods or bispecific antibodies (for use), medicaments or pharmaceutical formulations of the disclosure, the HRF volume in the central 1-mm diameter of the inner retina after 48 weeks of treatment is less than about 0.3 relative to the HRF volume in the central 1-mm diameter of the inner retina prior to treatment. In certain other embodiments, the HRF volume in the central 3-mm diameter of the inner retina after 48 weeks of treatment is less than about 0.5 relative to the HRF volume in the central 3-mm diameter of the inner retina prior to treatment.

“Diabetic Macular Edema” (DME), as used herein, refers to a serious eye condition that affects people with diabetes (type 1 or 2). Macular edema occurs when blood vessels in the retina leak into the macula and fluid and protein deposits collect on or under the macula of the eye and causes it to thicken and swell (edema). The swelling may distort a person's central vision, as the macula is near the center of the retina at the back of the eyeball. The primary symptoms of DME include, but are not limited to, blurry vision, floaters, loss of contrast, double vision, and eventual loss of vision. The pathology of DME is characterized by breakdown of inner the blood-retinal barrier, normally preventing fluid movement in the retina, thus allowing fluid to accumulate in the retinal tissue, and presence of retinal thickening. DME is presently diagnosed during an eye examination consisting of a visual acuity test, which determines the smallest letters a person can read on a standardized chart, a dilated eye exam to check for signs of the disease, imaging tests such as optical coherence tomography (OCT) or fluorescein angiography (FA) and tonometry, an instrument that measures pressure inside the eye. The following studies are also performed to determine treatment: optical coherence tomography (OCT), fluorescein angiography, and color stereo fundus photography. DME can be broadly characterized into two main categories—Focal and Diffuse. Focal DME is characterized by specific areas of separate and distinct leakage in the macula with sufficient macular blood flow. Diffuse DME results from leakage of the entire capillary bed surrounding the macula, resulting from a breakdown of the inner blood-retina barrier of the eye. In addition to Focal and Diffuse, DME is also categorized based on clinical exam findings into clinically significant macular edema (CSME), non-CSME and CSME with central involvement (CSME-CI), which involves the fovea. The present invention includes methods to treat the above-mentioned categories of DME.

As used herein, the term “a patient suffering from” may include a subset of population which is more susceptible to DME or AMD or may show an elevated level of a DME-associated or an AMD-associated biomarker. For example, “a subject in need thereof” may include a subject suffering from diabetes for more than 10 years, have frequent high blood sugar levels or high fasting blood glucose levels. In certain embodiments, the term “a patient suffering from” includes a subject who, prior to or at the time of administration of the bispecific anti-VEGF/ANG2 antibody, has or is diagnosed with diabetes. In certain embodiments, the term “a patient suffering from” includes a subject who, prior to or at the time of administration of the anti-VEGF/ANG2 antibody, is more than 50 years old. In some embodiments, the term “a patient suffering from” includes subjects who are smokers, or subjects with high blood pressure or high cholesterol.

In certain embodiments of the methods or bispecific antibodies (for use), medicaments or pharmaceutical formulations of the disclosure, the patient, prior to treatment with the bispecific antibody, has HRF in a volume of at least 1100 picoliters (pL) in the central 3-mm diameter of the inner retina, and/or at least 210 pL in the central 1-mm diameter of the inner retina, as measured by spectral-domain optical coherence tomography.

In certain embodiments, the patient, prior to treatment with the bispecific antibody, has HRF in a volume of at least 1400 pL in the central 3-mm diameter of the outer retina, and/or at least 150 pL in the central 1-mm diameter of the outer retina as measured by spectral-domain optical coherence tomography.

The present invention includes methods or bispecific antibodies (for use), medicaments or pharmaceutical formulations comprising administering a therapeutically effective amount of a bispecific anti-VEGF/ANG2 antibody (or a medicament or pharmaceutical formulation comprising the bispecific anti-VEGF/ANG2 antibody) to a subject in need thereof.

In certain embodiments of the methods or bispecific antibodies (for use), medicaments or pharmaceutical formulations of the disclosure,

In certain embodiments, the bispecific antibody, medicament or pharmaceutical formulation comprising such bispecific anti-VEGF/ANG2 antibody is administered (intravitreally) to the subject in multiple doses, e.g., as part of a specific therapeutic dosing regimen.

In certain embodiments of the methods or bispecific antibodies (for use), medicaments or pharmaceutical formulations of the disclosure, the dosing interval is shortened if HRF volume and/or count is not reduced relative to the HRF volume and/or count prior to treatment. In certain embodiments, the dosing interval is extended if HRF volume and/or count is reduced relative to the HRF volume and/or count prior to treatment. In certain other embodiments, the dosing interval is extended if HRF volume after 48 weeks of treatment is less than 0.5 relative to the HRF volume prior to treatment.

In certain embodiments of the methods or bispecific antibodies (for use), medicaments or pharmaceutical formulations of the disclosure, reducing HRF prolongs the time to retreatment and/or prolongs the time to loss of visual acuity (e.g., reduces the progression and/or severity of the disease).

In one embodiment of the methods or bispecific antibodies (for use), medicaments or pharmaceutical formulations of the disclosure, the bispecific antibody is administered in a dose of about 5 to 7 mg (at each treatment). In one embodiment the bispecific antibody is administered in a dose of 6 mg+/−10% (at each treatment). In one embodiment the bispecific antibody is administered in a dose of about 6 mg (at each treatment).

In certain embodiments of the methods or bispecific antibodies (for use), medicaments or pharmaceutical formulations of the disclosure, the bispecific antibody is administered every 8 weeks or less frequently (e.g., every 2 months). For example, the bispecific antibody is administered every 9 weeks or less frequently, every 10 weeks or less frequently, every 11 weeks or less frequently, every 12 weeks or less frequently (e.g., every 3 months), every 13 weeks or less frequently, every 14 weeks or less frequently, every 15 weeks or less frequently, every 16 weeks or less frequently (e.g., every 4 months).

In certain embodiments, the bispecific antibody is administered every 8 to 10 weeks, every 10 to 12 weeks, every 11 to 13 weeks, every 12 to 14 weeks, every 13 to 15 weeks, or every 14 to 16 weeks.

Such method, use, bispecific antibody (for use), medicament or pharmaceutical formulation may comprise sequentially administering initial doses (“treatment initiation”) (e.g. 3 to 7 monthly administrations; in one embodiment the treatment initiation includes 3 to 4 monthly administrations, in one embodiment the treatment initiation includes 4 to 5 monthly administrations; in one embodiment the treatment initiation includes 4 to 6 monthly administrations; in one embodiment the treatment initiation includes at least 4 monthly administrations; in one embodiment the treatment initiation includes 5 to 7 monthly administrations, in one embodiment the treatment initiation includes 6 monthly administrations) followed by one or more secondary doses of a therapeutically effective amount of the bispecific antibody, medicament or pharmaceutical formulation.

In certain embodiments of the methods or bispecific antibodies (for use), medicaments or pharmaceutical formulations of the disclosure, the bispecific antibody is administered following a treatment initiation, wherein the treatment initiation comprises 3 to 7 monthly (e.g., every 4 weeks) administrations.

In one embodiment, the bispecific antibody, medicament or pharmaceutical formulation is administered every 10 to 12 weeks (following treatment initiation). In one embodiment, the bispecific antibody, medicament or pharmaceutical formulation is administered every 11 to 13 weeks (following treatment initiation). In one embodiment, the bispecific antibody, medicament or pharmaceutical formulation is administered every 12 to 14 weeks (following treatment initiation). In one embodiment, the bispecific antibody, medicament or pharmaceutical formulation is administered every 13 to 15 weeks (following treatment initiation). In one embodiment, the bispecific antibody, medicament or pharmaceutical formulation is administered every 14 to 16 weeks (following treatment initiation).

In certain embodiments of the methods or bispecific antibodies (for use), medicaments or pharmaceutical formulations of the disclosure, the bispecific antibody is administered at a concentration of about 110 to 130 mg/mL. In certain embodiments, the bispecific antibody is administered at a concentration of about 120 mg/mL.

The bispecific antibody of the disclosure may be administered in a liquid pharmaceutical formulation. Suitable liquid formulation is disclosed in International Patent Application Publication No. WO2020/089051, which is incorporated herein in its entirety.

For example, in certain embodiments, the liquid pharmaceutical formulation comprises:
about 110 to 130 mg/mL of the bispecific antibody comprising,
about 15 to 35 mM of sodium, and
about 15 to 25 mM of a histidine acetate buffer, and having a pH of 5.5±0.5.

In certain embodiments, the liquid pharmaceutical formulation further comprises one or more of:
about 7.0 mM±2.0 mM methionine;
about 0.03% to 0.07% (w/v) polysorbate 20; and
about 160 mM±24 mM sucrose.

Such liquid pharmaceutical formulation, in certain embodiments, has a viscosity of about 20 mPas or less, and/or a turbidity of about 30 FTU or less, and/or an ionic strength between about 20 and 50. International Patent Application Publication No. WO2020/089051, which is incorporated herein in its entirety, describes suitable methods to determine viscosity, turbidity and ionic strength. In certain embodiments, the liquid pharmaceutical formulation is essentially free of visible particles. In certain other embodiments, the liquid pharmaceutical formulation is essentially free of (or does not comprise) calcium chloride and/or arginine.

Antibody specificity refers to selective recognition of the antibody for a particular epitope of an antigen. Natural antibodies, for example, are monospecific.

“Bispecific antibodies” according to the invention are antibodies which have two different antigen-binding specificities. Antibodies of the present invention are specific for two different antigens, VEGF as first antigen and ANG-2 as second antigen.

The term “monospecific” antibody as used herein denotes an antibody that has one or more binding sites each of which bind to the same epitope of the same antigen.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. The bispecific antibodies according to the invention are preferably “bivalent”.

The terms “bispecific antibody which binds to human vascular endothelial growth factor (VEGF) and to human angiopoietin-2 (ANG-2)”, “bispecific anti-VEGF/ANG2 antibody” and “bispecific <VEGF/ANG2> antibody” as used herein are interchangeable and refer to an antibody which has at least two different antigen-binding sites, a first one which binds to VEGF and a second one which binds to ANG2.

Bispecific anti-VEGF/ANG2 antibodies are e.g. described in International Patent Application Publication Nos. WO2010/040508, WO2011/117329, WO2012/131078, WO2015/083978, WO2017/197199, and WO2014/009465. WO2014/009465 describes bispecific anti-VEGF/ANG2 antibodies especially designed for treatment of ocular vascular diseases. The bispecific anti-VEGF/ANG2 antibodies of WO2014/009465 (which is incorporated herein in its entirety) are especially useful in the treatment and treatment schedules of ocular vascular diseases as described herein. In particular, anti-VEGF/ANG2 antibody CrossMAb VEGFang2-0016 as described in WO2014/009465 which is also described as faricimab (in World Health Organization (2017). “International Nonproprietary Names for Pharmaceutical Substances (INN). Proposed INN: List 118” WHO Drug Information. 31 (4)) is a preferred bispecific anti-VEGF/ANG2 antibody of the present invention.

In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO: 6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations 1253A, H310A, and H435A and the mutations L234A, L235A and P329G (numberings according to EU Index of Kabat).

In one embodiment such bispecific anti-VEGF/ANG2 antibody is bivalent.

In one embodiment such bispecific, bivalent anti-VEGF/ANG2 antibody is characterized in that
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

In one aspect of the invention such bispecific, bivalent antibody according to the invention is characterized in comprising
a) the heavy chain and the light chain of a first full length antibody that specifically binds to VEGF;
b) the modified heavy chain and modified light chain of a second full length antibody that specifically binds to ANG-2, wherein the constant domains CL and CH1 are replaced by each other.

This bispecific, bivalent antibody format for the bispecific antibody specifically binding to human vascular endothelial growth factor (VEGF) and human angiopoietin-2 (ANG-2) is described in WO 2009/080253 (including Knobs-into-Holes modified CH3 domains). The antibodies based on this bispecific, bivalent antibody format are named CrossMAbs.

In one embodiment such bispecific, bivalent anti-VEGF/ANG2 antibody is characterized in comprising:
a) as heavy chain of the first full length antibody the amino acid sequence of SEQ ID NO: 17, and as light chain of the first full length antibody the amino acid sequence of SEQ ID NO: 18, and
b) as modified heavy chain of the second full length antibody the amino acid sequence of SEQ ID NO: 19, and as modified light chain of the second full length antibody the amino acid sequence of SEQ ID NO: 20.

In one embodiment such bispecific, bivalent anti-VEGF/ANG2 antibody is characterized in comprising the amino acid sequences of SEQ ID NO: 17, of SEQ ID NO: 18, of SEQ ID NO: 19, and of SEQ ID NO: 20.

Accordingly, one embodiment of the invention is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20.

In one embodiment the CH3 domains of the bispecific, bivalent antibody according to the invention is altered by the “knob-into-holes” technology which is described in detail with several examples in e.g. WO 96/027011, Ridgway J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M, et al., Nature Biotech 16 (1998) 677-681; Atwell, S., et al. J. Mol. Biol. 270 (1997) 26-35) and increases the yield.

In a preferred aspect of the invention the bispecific anti-VEGF/ANG2 antibodies according to the invention are characterized in that the CH3 domain of one heavy chain and the CH3 domain of the other heavy chain each meet at an interface which comprises an original interface between the antibody CH3 domains;
wherein said interface is altered to promote the formation of the bispecific antibody, wherein the alteration is characterized in that:
a) the CH3 domain of one heavy chain is altered,
so that within the original interface the CH3 domain of one heavy chain that meets the original interface of the CH3 domain of the other heavy chain within the bispecific antibody,
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and
b) the CH3 domain of the other heavy chain is altered,
so that within the original interface of the second CH3 domain that meets the original interface of the first CH3 domain within the bispecific antibody
an amino acid residue is replaced with an amino acid residue having a smaller side chain volume, thereby generating a cavity within the interface of the second CH3 domain within which a protuberance within the interface of the first CH3 domain is positionable.

Thus the bispecific anti-VEGF/ANG2 antibodies for use described herein are preferably characterized in that
the CH3 domain of the heavy chain of the full length antibody of a) and the CH3 domain of the heavy chain of the full length antibody of b) each meet at an interface which comprises an alteration in the original interface between the antibody CH3 domains;
wherein i) in the CH3 domain of one heavy chain
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and wherein
ii) in the CH3 domain of the other heavy chain
an amino acid residue is replaced with an amino acid residue having a smaller side chain volume, thereby generating a cavity within the interface of the second CH3 domain within which a protuberance within the interface of the first CH3 domain is positionable.

Preferably said amino acid residue having a larger side chain volume is selected from the group consisting of arginine (R), phenylalanine (F), tyrosine (Y), tryptophan (W).

Preferably said amino acid residue having a smaller side chain volume is selected from the group consisting of alanine (A), serine (S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed.

In one embodiment, the bispecific antibody comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M, et al., Nature Biotech 16 (1998) 677-681) e.g. by introducing a S354C mutation into one CH3 domain and a Y349C mutation into the other CH3 domain.

In a another preferred embodiment the bispecific antibody comprises S354C and T366W mutations in one of the two CH3 domains and Y349C, T366S, L368A, Y407V mutations in the other of the two CH3 domains In a another preferred embodiment the bispecific antibody comprises Y349C, T366W mutations in one of the two CH3 domains and S354C, T366S, L368A, Y407V mutations in the other of the two CH3 domains (the additional Y349C or S354C mutation in one CH3 domain and the additional S354C or Y349C mutation in the other CH3 domain forming a interchain disulfide bridge) (numbering always according to EU index of Kabat (Kabat, E. A., et al., Sequences of Proteins of Immunological Interest, 5th ed., Public Health Service, National Institutes of Health, Bethesda, MD (1991)).

Other techniques for CH3-modifications to enforce the heterodimerization are contemplated as alternatives of the invention and described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291.

In one embodiment the heterodimerization approach described in EP 1 870 459A1 is used alternatively. This approach is based on the introduction of substitutions/mutations of charged amino acids with the opposite charge at specific amino acid positions of the in the CH3/CH3 domain interface between both heavy chains. One preferred embodiment for said multispecific antibodies are amino acid R409D and K370E mutations in the CH3 domain of one heavy chain and amino acid D399K and E357K mutations in the CH3 domain of the other heavy chain of the multispecific antibody (numberings according to Kabat EU index).

In another embodiment said multispecific antibody comprises an amino acid T366W mutation in the CH3 domain of the “knobs chain” and amino acid T366S, L368A and Y407V mutations in the CH3 domain of the “hole chain”; and additionally comprises amino acid R409D and K370E mutations in the CH3 domain of the “knobs chain” and amino acid D399K and E357K mutations in the CH3 domain of the “hole chain”.

In one embodiment the heterodimerization approach described in WO2013/157953 is used alternatively. In one embodiment the CH3 domain of one heavy chain comprises an amino acid T366K mutation and the CH3 domain of the other heavy chain comprises an amino acid L351D mutation. In a further embodiment the CH3 domain of the one heavy chain further comprises an amino acid L351K mutation. In a further embodiment the CH3 domain of the other heavy chain further comprises an amino acid mutation selected from Y349E, Y349D and L368E (in one embodiment L368E).

In one embodiment the heterodimerization approach described in WO2012/058768 is used alternatively. In one embodiment the CH3 domain of one heavy chain comprises amino acid L351Y and Y407A mutations and the CH3 domain of the other heavy chain comprises amino acid T366A and K409F mutations. In a further embodiment the CH3 domain of the other heavy chain further comprises an amino acid mutation at position T411, D399, S400, F405, N390 or K392. In one embodiment said amino acid mutation is selected from the group consisting of
a) T41N, T411R, T41Q, T411K, T41D, T411E and T411W,
b) D399R, D399W, D399Y and D399K,
c) S400E, S400D, S400R and S400K,
d) F405I, F405M, F405T, F405S, F405V and F405W,
e) N390R, N390K and N390D,
f) K392V, K392M, K392R, K392L, K392F and K392E.

In a further embodiment the CH3 domain of one heavy chain comprises amino acid L351Y and Y407A mutations and the CH3 domain of the other heavy chain comprises amino acid T366V and K409F mutations. In a further embodiment the CH3 domain of one heavy chain comprises an amino acid Y407A mutation and the CH3 domain of the other heavy chain comprises amino acid T366A and K409F mutations. In a further embodiment the CH3 domain of the other heavy chain further comprises amino acid K392E, T411E, D399R and S400R mutations.

In one embodiment the heterodimerization approach described in WO2011/143545 is used alternatively. In one embodiment the amino acid modification according to WO2011/143545 is introduced in the CH3 domain of the heavy chain at a position selected from the group consisting of 368 and 409.

In one embodiment the heterodimerization approach described in WO2011/090762 which also uses the knob-into-hole technology described above is used alternatively. In one embodiment the CH3 domain of one heavy chain comprises an amino acid T366W mutation and the CH3 domain of the other heavy chain comprises an amino acid Y407A mutation. In one embodiment the CH3 domain of one heavy chain comprises an amino acid T366Y mutation and the CH3 domain of the other heavy chain comprises an amino acid Y407T mutation.

In one embodiment the multispecific antibody is of IgG2 isotype and the heterodimerization approach described in WO2010/129304 is used alternatively.

In one embodiment the heterodimerization approach described in WO2009/089004 is used alternatively. In one embodiment the CH3 domain of one heavy chain comprises an amino acid substitution of K392 or N392 with a negatively-charged amino acid (in one embodiment glutamic acid (E) or aspartic acid (D); in a further embodiment a K392D or N392D mutation) and the CH3 domain of the other heavy chain comprises an amino acid substitution of D399, E356, D356, or E357 with a positively-charged amino acid (in one embodiment Lysine (K) or arginine (R), in a further embodiment a D399K, E356K, D356K or E357K substitution; and in an even further embodiment a D399K or E356K mutation). In a further embodiment the CH3 domain of the one heavy chain further comprises an amino acid substitution of K409 or R409 with a negatively-charged amino acid (in one embodiment glutamic acid (E) or aspartic acid (D); in a further embodiment a K409D or R409D mutation). In a further embodiment the CH3 domain of the one heavy chain further or alternatively comprises an amino acid substitution of K439 and/or K370 with a negatively-charged amino acid (in one embodiment glutamic acid (E) or aspartic acid (D)).

In one embodiment the heterodimerization approach described in WO2007/147901 is used alternatively. In one embodiment the CH3 domain of one heavy chain comprises amino acid K253E, D282K and K322D mutations and the CH3 domain of the other heavy chain comprises amino acid D239K, E240K and K292D mutations.

In one embodiment the heterodimerization approach described in WO2007/110205 is used alternatively.

In one preferred embodiment such bispecific anti-VEGF/ANG2 antibody is bivalent.

In one embodiment the bispecific, bivalent antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO: 6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat; and wherein
iv) in the constant heavy chain region a T366W mutation is comprised in one CH3 domain and T366S, L368A, Y407V mutations are comprised the other CH3 domain (numberings according to EU Index of Kabat).

In one embodiment the bispecific, bivalent antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO: 6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat; and wherein
iv) in the constant heavy chain region a S354C and T366W mutations are comprised in one CH3 domain and Y349C, T366S, L368A and Y407V mutations are comprised the other CH3 domain (numberings according to EU Index of Kabat).

In one embodiment such bispecific, bivalent anti-VEGF/ANG2 is characterized in comprising the amino acid sequences of SEQ ID NO: 17, of SEQ ID NO: 18, of SEQ ID NO: 19, and of SEQ ID NO: 20.

Accordingly, one embodiment of the invention is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20.

In one preferred embodiment such bispecific anti-VEGF/ANG2 antibody is faricimab.

The term “VEGF” as used herein refers to human vascular endothelial growth factor (VEGF/VEGF-A,) the 165-amino acid human vascular endothelial cell growth factor (amino acid 27-191 of precursor sequence of human VEGF165: SEQ ID NO: 25; amino acids 1-26 represent the signal peptide), and related 121, 189, and 206 vascular endothelial cell growth factor isoforms, as described by Leung, D. W., et al., Science 246 (1989) 1306-9; Houck et al., Mol. Endocrin. 5 (1991) 1806-1814; Keck, P. J., et al., Science 246 (1989) 1309-12 and Connolly, D. T., et al., J. Biol. Chem. 264 (1989) 20017-24; together with the naturally occurring allelic and processed forms of those growth factors. VEGF is involved in the regulation of normal and abnormal angiogenesis and neovascularization associated with tumors and intraocular disorders (Ferrara, N., et al., Endocr. Rev. 18 (1997) 4-25; Berkman, R. A., et al., J. Clin. Invest. 91 (1993) 153-159; Brown, L. F., et al., Human Pathol. 26 (1995) 86-91; Brown, L. F., et al., Cancer Res. 53 (1993) 4727-4735; Mattern, J., et al., Brit. J. Cancer. 73 (1996) 931-934; and Dvorak, H. F., et al., Am. J. Pathol. 146 (1995) 1029-1039). VEGF is a homodimeric glycoprotein that has been isolated from several sources and includes several isoforms. VEGF shows highly specific mitogenic activity for endothelial cells. A VEGF antagonist/inhibitor inhibits binding of VEGF to its receptor VEGFR. Known VEGF antagonist/inhibitors include bispecific anti-VEGF/ANG2 antibodies as described in WO2014/009465.

The term “ANG-2” as used herein refers to human angiopoietin-2 (ANG-2) (alternatively abbreviated with ANGPT2 or ANG2) (SEQ ID NO: 24) which is described e.g. in Maisonpierre, P. C., et al, Science 277 (1997) 55-60 and Cheung, A. H., et al., Genomics 48 (1998) 389-91. The angiopoietins-1 and -2 were discovered as ligands for the Ties, a family of tyrosine kinases that is selectively expressed within the vascular endothelium (Yancopoulos, G. D., et al., Nature 407 (2000) 242-48). There are now four definitive members of the angiopoietin family. Angiopoietin-3 and -4 (Ang-3 and Ang-4) may represent widely diverged counterparts of the same gene locus in mouse and man (Kim, I., et al., FEBS Let, 443 (1999) 353-56; Kim, I., et al., J Biol Chem 274 (1999) 26523-28). ANG-1 and ANG-2 were originally identified in tissue culture experiments as agonist and antagonist, respectively (see for ANG-1: Davis, S., et al., Cell 87 (1996) 1161-69; and for ANG-2: Maisonpierre, P. C., et al., Science 277 (1997) 55-60). All of the known angiopoietins bind primarily to its receptor TIE2, and both Ang-1 and -2 bind to TIE2 with an affinity of 3 nM (Kd) (Maisonpierre, P. C., et al., Science 277 (1997) 55-60). An ANG2 antagonist/inhibitor inhibits binding of ANG2 to its receptor TIE2. Known ANG2 antagonist/inhibitors include bispecific anti-VEGF/ANG2 antibodies as described in WO2014/009465.

An antigen-binding sites of the bispecific antibody of the invention contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences.

The antibodies of the invention comprise immunoglobulin constant regions derived from human origin of immunoglobulin class IgG1.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are of particular interest. Other forms of “chimeric antibodies” encompassed by the present invention are those in which the constant region has been modified or changed from that of the original antibody to generate the desired properties according to the invention, especially in regard to C1q binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See e.g. Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boemer et al. are also available for the preparation of human monoclonal antibodies (Cole et al., Monoclonal Antibodies and Cancer Therapy, Alan R. Liss, p. 77 (1985); and Boemer, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation.).

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable region” (variable region of a light chain (V_{L}), variable region of a heavy chain (V_{H})) or “variable domain” as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a R-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention. The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, MD (1991) and/or those residues from a “hypervariable loop”.

The term “epitope” includes any polypeptide determinant capable of specific binding to an antibody. In certain embodiments, epitope determinant includes chemically active surface groupings of molecules such as amino acids, sugar side chains, phosphoryl, or sulfonyl, and, in certain embodiments, may have specific three-dimensional structural characteristics, and or specific charge characteristics. An epitope is a region of an antigen that is bound by an antibody.

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be kappa or lambda. The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG1 and IgG2), IgM, IgA, IgD, and IgE. The full length antibodies according to the invention can be from a single species e.g. human, or they can be chimerized or humanized antibodies. The full length antibodies according to the invention comprise two antigen binding sites each formed by a pair of VH and VL, which both specifically bind to the same antigen. The C-terminus of the heavy or light chain of said full length antibody denotes the last amino acid at the C-terminus of said heavy or light chain. The N-terminus of the heavy or light chain of said full length antibody denotes the last amino acid at the N-terminus of said heavy or light chain.

The term “constant region” or “constant domains” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called alpha, delta., epsilon., gamma, and micro, respectively. The light chain constant regions which can be found in all five antibody classes are called kappa and lambda.

The term “constant region derived from human origin” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., (see e.g. Johnson, G., and Wu, T. T., Nucleic Acids Res. 28 (2000) 214-218; Kabat, E. A., et al, Proc. Natl. Acad. Sci. USA 72 (1975) 2785-2788).

The term constant heavy chain domain (or region) as used herein defines a C-terminal region of an immunoglobulin heavy chain that contains at least a portion of the constant heavy chain region.

The term includes native sequences of the constant heavy chain domains and variant constant heavy chain domains. Variant constant heavy chain domains include e.g. mutations in the constant domain which are used to foster the heterodimerization as describe above for the knobs into hole technology. Also other mutations like e.g. L234A (Leu235Ala), L235A (Leu234Ala) and P329G (Pro329Gly) can be included as constant domains with such mutations have a reduced FcR binding (especially they show no more binding to FcRgammaI, FcRgammaII and FcRgammaIII). This especially useful to reduce potential side effects like e.g. thrombosis (Meyer, T., et al., J. Thromb. Haemost. 7 (2009) 171-81). In addition e.g. also the mutations 1253A, H310A, and H435A (numbering according to EU Index of Kabat) can be included in the constant domain as constant domains with such mutations have a reduced FcRn one or two mutations) or eliminated FcRn binding (all 3 mutations).

In one aspect, a human IgG heavy chain constant region extends from alanine118 (A118) (numbering according to EU index of Kabat) to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the constant heavy chain domain may or may not be present. Amino acid sequences of heavy chains including the constant heavy chain domain are denoted herein with C-terminal glycine-lysine dipeptide if not indicated otherwise.

In one embodiment the bispecific antibodies according to the invention have a constant region of human IgG1 subclass (derived from human IgG1 subclass). However, the C-terminal lysine (Lys447), or the C-terminal glycine (Gly446) and the C-terminal lysine (Lys447), of the Fc region may or may not be present.

In one embodiment the bispecific antibody as described herein is of IgG1 isotype/subclass and comprises a constant heavy chain domain of SEQ ID NO: 23 or the constant parts of the heavy chain amino acid sequence of SEQ ID NO: 17 and of the heavy chain amino acid sequence of SEQ ID NO: 18. In one embodiment additionally the C-terminal glycine (Gly446) is present. In one embodiment additionally the C-terminal glycine (Gly446) and the C-terminal lysine (Lys447) is present.

Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

In one embodiment the bispecific antibody according to the invention is of human IgG1 subclass with mutations L234A (Leu235Ala), L235A (Leu234Ala) and P329G (Pro329Gly). Such antibody has a reduced FcR binding (especially they show no more binding to FcRgammaI, FcRgammaII and FcRgammaIII). This especially useful to reduce potential side effects like e.g. thrombosis (Meyer, T., et al., J. Thromb. Haemost. 7 (2009) 171-81).

While Pro329Ala mutation which was described already removes only two third of the FcgammaRIIIa sandwich interaction, the Pro329Gly in the antibodies according to the invention fully imparts binding of the Fc part to FcgammaRIII. This is especially useful as the binding to FcgammaRIII is involved in ADCC (antibody-dependent cellular toxicity) which leads to cell death, which may be helpful in the treatment of cancer diseases, but which can cause serious side effect in the antibody based treatment of other vascular or immunological diseases. So the antibodies according to the invention of IgG1 subclass with mutations L234A, L235A and P329G and IgG4 subclass with mutations S228P, L235E and P329G are especially useful, as they both show no more binding to FcRgammaI, FcRgammaII and FcRgammaIII.

An “effective amount” of an agent, e.g., a pharmaceutical formulation or bispecific anti-VEGF/ANG2 antibody, refers to an amount effective, at dosages and for periods of time necessary, to achieve the desired therapeutic or prophylactic result.

In one embodiment of the invention the bispecific antibody, medicament or pharmaceutical formulation as described herein is administered via intravitreal application, e.g. via intravitreal injection (is administered “intravitreally”). This can be performed in accordance with standard procedures known in the art. See, e.g., Ritter et al., J. Clin. Invest. 116 (2006) 3266-76; Russelakis-Cameiro et al., Neuropathol. Appl. Neurobiol. 25 (1999) 196-206; and Wray et al., Arch. Neurol. 33 (1976) 183-5.

In some embodiments, therapeutic kits of the invention can contain one or more doses of the bispecific antibody described present in a medicament or pharmaceutical formulation, a suitable device for intravitreal injection of the medicament or pharmaceutical formulation, and an instruction detailing suitable subjects and protocols for carrying out the injection. In these embodiments, the medicament or pharmaceutical formulation are typically administered to the subject in need of treatment via intravitreal injection. This can be performed in accordance with standard procedures known in the art. See, e.g., Ritter et al., J. Clin. Invest. 116 (2006) 3266-76; Russelakis-Carneiro et al., Neuropathol. Appl. Neurobiol. 25 (1999) 196-206; and Wray et al., Arch. Neurol. 33 (1976) 183-5.

Regardless of the route of administration selected, the bispecific antibody as described herein is formulated into pharmaceutically acceptable dosage forms by conventional methods known to those of skill in the art.

The compositions and methods of the disclosure are illustrated further by the following example, which is not to be construed as limiting the disclosure in scope or spirit to the specific methods and compositions described in them.

### EXAMPLE

### Reduction in Hyperreflective Foci in Diabetic Macular Edema (DME)

### Materials and Methods:

The aim of this example is to assess whether faricimab has a greater impact on reducing HRF within the retina of DME patients compared to aflibercept for patients enrolled in two phase 3 clinical trials, YOSEMITE (ClinicalTrials.gov identifier: NCT03622580) and RHINE (ClinicalTrials.gov identifier: NCT03622593) (Wykoff C C, et al. Efficacy, durability, and safety of intravitreal faricimab with extended dosing up to every 16 weeks in patients with diabetic macular oedema (YOSEMITE and RHINE): two randomized, double-masked, phase 3 trials. The Lancet. 2022; 399(10326):741-755; Eter N, et al. YOSEMITE and RHINE: Phase 3 Randomized Clinical Trials of Faricimab for Diabetic Macular Edema: Study Design and Rationale. Ophthalmol Sci. 2022;2(1):100111.).

The YOSEMITE and RHINE trials were 2 identically designed, double-masked, multicenter, randomized, parallel-group, registrational phase 3 studies of faricimab in patients with DME. The studies were designed to evaluate the efficacy, safety, pharmacokinetics, and durability of intravitreal faricimab 6.0 mg for the treatment of DME when dosed either every 8 weeks or according to a PTI regimen in adjustable intervals (up to every 16 weeks), compared with intravitreal aflibercept 2.0 mg dosed every 8 weeks as per the label.

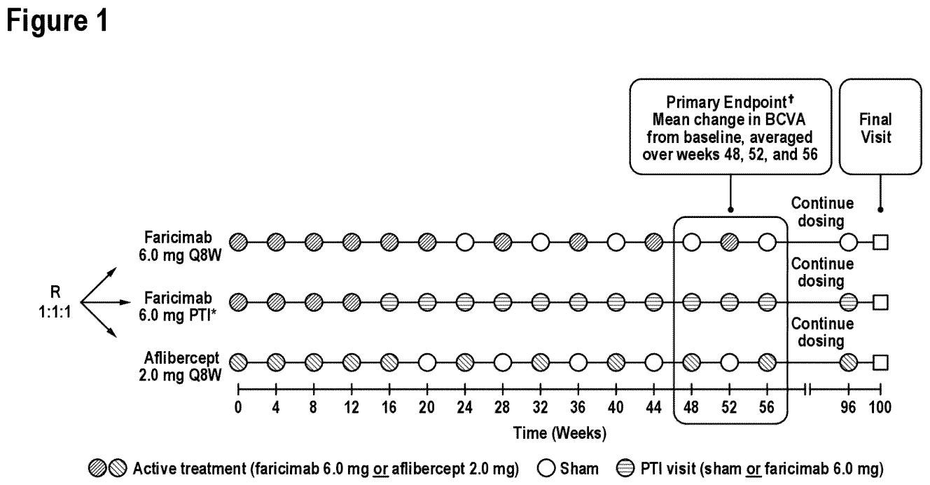

The YOSEMITE and RHINE trials enrolled 1891 patients (YOSEMITE, 940 patients; RHINE, 951 patients). The studies comprised 3 treatment arms: (1) faricimab 6.0 mg monthly (every 4 weeks, Q4W) for 6 months followed by every-8-week dosing (Q8W); (2) faricimab 6.0 mg every 4 weeks for 4 months followed by per PTI, a protocol-driven T&E regimen with up to every-16-week dosing; or (3) aflibercept 2.0 mg every 4 weeks (Q4W) for 5 months followed by every-8-week dosing (Q8W), in line with the product label (FIG. 1). Patients were randomized 1:1:1 to each of the 3 treatment arms of the studies (FIG. 1). Randomization was stratified by baseline best-corrected visual acuity (BCVA) Early Treatment Diabetic Retinopathy Study (ETDRS) letter score (64 ETDRS letters or better vs. 63 letters or worse; Snellen equivalent threshold, ˜20/63), prior intravitreal anti-VEGF therapy (yes vs. no), and region (United States and Canada, Asia, and the rest of the world). The goal of stratification was to prevent imbalance of these potentially confounding variables across the study arms that could affect the interpretation of study outcomes. To preserve masking, patients were seen every 4 weeks and underwent a sham procedure at study treatment visits when they were not treated with active study drug. For the purpose of the current example, YOSEMITE and RHINE data were pooled.

### Study Participants and Eligibility Criteria

Patients 18 years of age or older with center-involving DME secondary to type 1 or type 2 diabetes mellitus were eligible to participate. The inclusion criterion for hemoglobin A1c level was set at up to 10% to limit enrollment of patients with unstable diabetic control to minimize any potential changes to the outcome variables that could be secondary to large fluctuations in underlying glucose levels. General exclusion criteria included, among others, untreated diabetes or treatment initiated within 3 months of day 1; uncontrolled high blood pressure; and history of other disease, physical examination finding, or clinical laboratory finding suggestive of a condition that would contraindicate use of any of the study drugs, may affect interpretation of the study results, or in the opinion of the investigator would render the patient at high risk for treatment complications.

One eye per patient was designated as the study eye. Ocular exclusion and inclusion criteria for the study eye are shown in Table 1. The central reading centers (CRCs) evaluated the spectral-domain (SD) OCT and color fundus photography (CFP) images obtained at screening to provide an objective, masked assessment of whether patients' study eyes met the study eligibility criteria. If both eyes were eligible for inclusion, the eye with the worse BCVA at screening was selected as the study eye.

TABLE 1
Ocular Exclusion and Inclusion Criteria for the Study Eye

| Exclusion Criteria | Inclusion Criteria |
|---|---|
| High-risk PDR in the study eye (any vitreous or | Macular thickening secondary to DME |
| preretinal hemorrhage; neovascularization | involving the center of the fovea, with |
| elsewhere one-half disc area or more within an | CST ≥325 μm (defined as the thickness from |
| area equivalent to the mydriatic ETDRS 7 fields | the ILM to Bruch's membrane), measured by |
| on clinical examination or on CFP images; | SD OCT or SS OCT (Spectralis |
| neovascularization at disc one-third disc area or | [Heidelberg Engineering GmbH, |
| more on clinical examination), as graded by the | Heidelberg, Germany], Topcon [Topcon, |
| CRCs | Tokyo, Japan], or Cirrus [Carl Zeiss |
| Tractional retinal detachment, preretinal | Meditec, Dublin, CA]) in the central 1-mm |
| fibrosis, or epiretinal membrane involving the | area of the macula as graded by the CRCs |
| fovea or disrupting the macular architecture in | BCVA between 25 and 73 ETDRS letters |
| the study eye | (approximate Snellen equivalent, 20/320- |
| Active rubeosis | 20/40), as assessed on the standardized |
| Uncontrolled glaucoma | ETDRS chart at 4 mos |
| History of retinal detachment or macular hole | Sufficiently clear ocular media and |
| (stage 3 or 4) | adequate pupillary dilatation to allow |
| Aphakia or implantation of anterior chamber | acquisition of good-quality CFP images |
| intraocular lens | (including ETDRS 7 modified fields or 4 |
| Intravitreal anti-VEGF treatment within 3 | wide-angle fields to permit grading of DR |
| months^{§} (previously treated patients) or any | and assessment of the retina) and other |
| intravitreal anti-VEGF agents in study eye | imaging methods |
| before day 1 (treatment-naive patients) | |
| Treatment with PRP within 3 mos^{§} | |
| Macular (focal or grid) laser within 3 mos^{§} | |
| Any cataract surgery or treatment for | |
| complications of cataract surgery with steroids | |
| or YAG laser capsulotomy within 3 mos^{§} | |
| Any other intraocular surgery | |
| Any intravitreal or periocular (sub-Tenon) | |
| corticosteroid treatment within 6 mos^{§} | |
| Any use of medicated intraocular implants, | |
| including Ozurdex (Allergan USA, Inc., | |
| Madison, NJ), within 6 mos^{§} | |
| Any use of Iluvien implants at any time | |
| Treatment for other retinal diseases that can | |
| lead to macular edema | |

PRP: panretinal photocoagulation; SD: spectral-domain; SS: swept-source; YAG: yttrium-aluminum-garnet.
^{§}Before day 1 of study.

Study eyes were permitted to be either anti-VEGF treatment naïve (with no previous history of intravitreal anti-VEGF therapy) or previously anti-VEGF treated (provided that the last treatment was ≥3 months before the day 1 study visit). Study eyes previously treated with anti-VEGF therapy were capped at 25% of the total patient enrollment for each study. The rationale for capping the number of patients previously treated with anti-VEGF therapy was based on the heterogeneous nature of this patient population, with a potential history of long-standing and potentially insufficiently treated DME, resulting in pharmacologically irreversible macular damage that could thus limit the possibility of visual acuity improvements.

### Data Selection

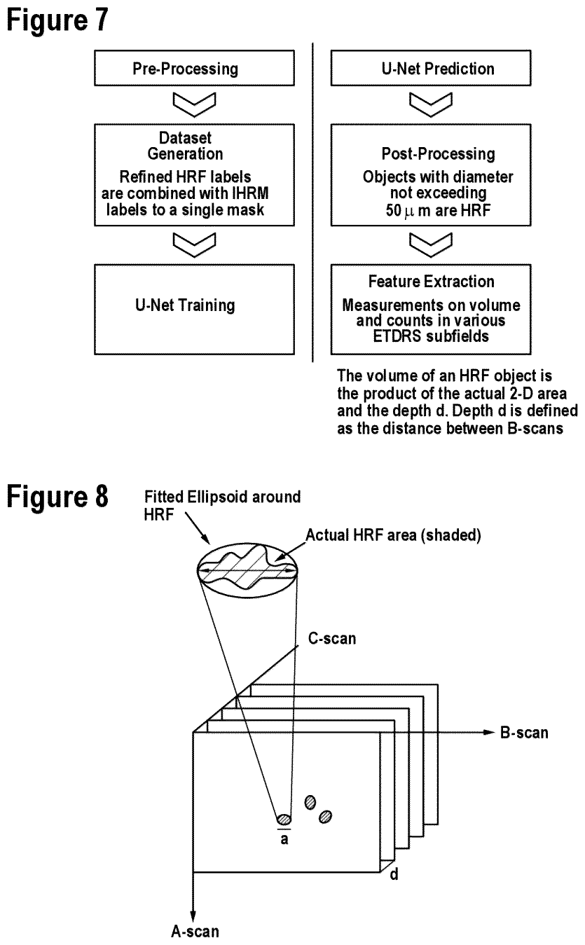

Subjects from phase 3 YOSEMITE and RHINE with both baseline and week 48 scans available were included in the analysis. Volumes acquired on Zeiss and Topcon devices, representing 17.8% and 0.1% of all volumes acquired, respectively, were excluded in order to obtain a homogenous data set of Spectralis (Heidelberg Engineering, Heidelberg, Germany) volumes. SD-OCT volume scans from the first year (baseline to week 48) were retrieved and all available volumes with 97 B-scans were segmented with the model described below (summarized in FIG. 7). A final set of 519, 524, and 502 patients treated with faricimab 6.0 mg Q8W, faricimab 6.0 mg T&E, and aflibercept 2.0 mg Q8W, respectively, was obtained, and all images from each available visit were analyzed.

### HRF Segmentation Model

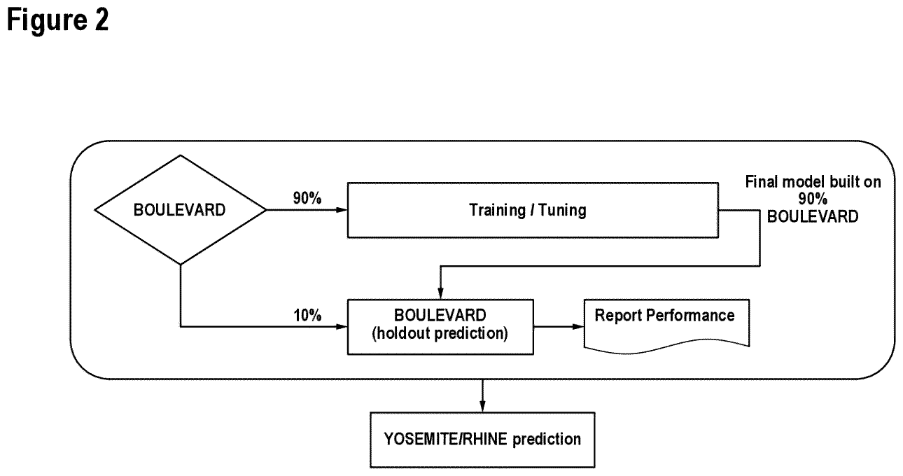

Overview. Manually annotated SD-OCT volume scans (Spectralis) from BOULEVARD to train and validate the HRF segmentation model were used. This model was subsequently applied to the YOSEMITE and RHINE SD-OCT volume scans (Spectralis) to generate the analyses presented (see FIG. 2). BOULEVARD (ClinicalTrials.gov identifier: NCT02699450) was a randomized, double-masked, phase 2 clinical trial, which compared the safety and efficacy of faricimab to ranibizumab in in treatment-naïve and previously treated patients (approximately 25% of study population) with centre-involving DME (Sahni J, et al. Simultaneous Inhibition of Angiopoietin-2 and Vascular Endothelial Growth Factor-A with Faricimab in Diabetic Macular Edema: BOULEVARD Phase 2 Randomized Trial. Ophthalmology. 2019; 126(8):1155-1170). Eyes were treated with faricimab 6.0 mg, faricimab 1.5 mg, or ranibizumab 0.3 mg every 4 weeks for 20 weeks, followed by 16 weeks of off-treatment observation.

HRF are distinct, bright dots on SD-OCT. A range of upper size limits have been reported in the literature, ranging from 30 to 50 μm. 50 μm was selected as the upper limit in order to also capture HRF that may be aggregated into objects larger than 30 μm.

Dataset and annotation. SD-OCT volumes (Spectralis) from BOULEVARD were selected post hoc and annotated on the B-scan level by two trained readers from the Liverpool Ophthalmic Reading Centre. Each B-scan was annotated by a single grader, and subselection of annotations from each grader was adjudicated and reviewed by a senior clinician. Per volume, two to nine B-scans were annotated by overlaying ellipses over HRF up to 50 μm in diameter, and manually outlining larger objects of intraretinal hyperreflective material (IHRM). Owing to the transverse resolution of 14 μm on SD-OCT, objects smaller than 20 μm were not annotated, due to the difficulty in differentiation. The following retinal-layer boundaries were also annotated, using single lines across each B-scan: Internal limiting membrane (ILM), boundary of outer plexiform layer-Henle's fiber layer (OPL-HFL), and center of retinal pigment epithelium (RPE).

Post-processing of annotations. Ellipses, shapes, and boundaries drawn on the B-scans were stored in raster format, then converted to label maps of the original image dimension. Disrupted layers were filtered out. Subsequently, ellipses were shrunk to the most hyperreflective center using adaptive thresholding, and combined with the IHRM shapes to form a single label of hyperreflective material. For the layers, each space between a pair of adjacent boundaries was filled with a distinct label.

Model training. The BOULEVARD data was split on the patient level into training (1355 B-scans) and validation (155 B-scans) sets. Training images and corresponding annotation masks were used to train the computer to recognize HRF and IHRM. Specifically, the multiclass U-Net, a convolutional neural network for biomedical-image segmentation, was trained for pixel-level semantic segmentation using 250 epochs, categorical Sorensen-Dice coefficient scores (DICE) loss, and Adam optimizer. Similarly, the layers were trained with 50 epochs.

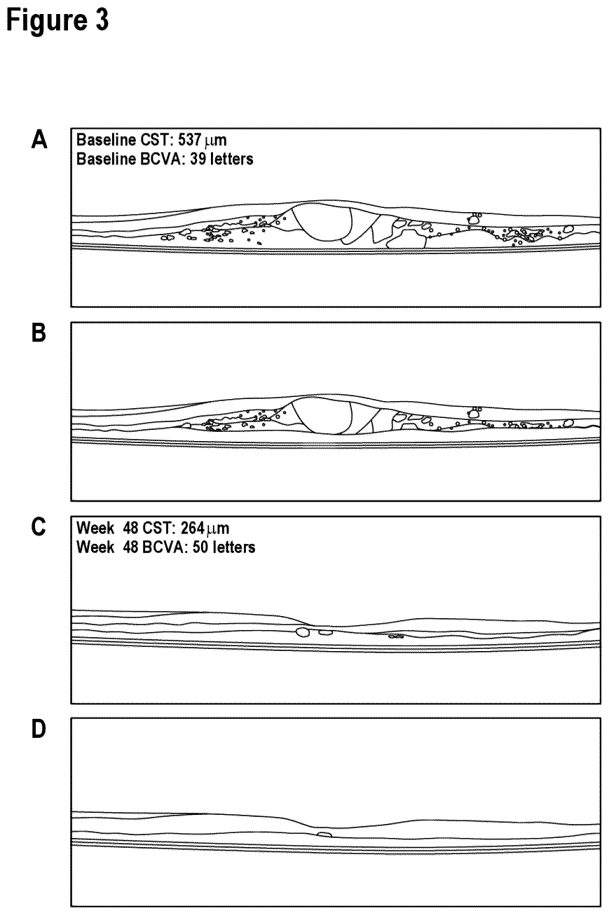

Post-processing of predictions. Objects detected by the model were categorized on the B-scan level by fitting an ellipse to each object (FIG. 8), and those below 50 μm in diameter (long axis) were classified as HRF. The remainder were discarded. HRF predictions were reviewed by a panel of experts to ensure that no noise was classified as HRF. Predicted layers were converted back to layer boundaries and stored in elevation-map format. Continuous layers were predicted regardless of potential disruptions. Example scans showing the automated prediction of HRF and layers are shown in FIG. 3.

Feature extraction. Using the B-scan-level predictions, two types of features were automatically extracted on the SD-OCT-volume level: Counts of distinct HRF objects across B-scans, and total HRF volume. Slice thickness, i.e., the space between the centers of two adjacent B-scans, was used as depth information to calculate volumes.

HRF counts and volumes were assessed in the 1-mm and 3-mm Early Treatment Diabetic Retinopathy Study (ETDRS) rings, which were also separated into inner and outer retina. The inner retina was defined as ILM to OPL-HFL, and the outer retina as OPL-HFL to RPE. The total retina was defined as ILM to RPE.

Both the 1-mm and 3-mm diameter rings were selected for analysis, as the former area is where retinal damage has the greatest impact on vision, and the latter covers the majority of the pathology. The 6-mm diameter was excluded from the analysis due to significant variability in scan area for Spectralis volume scans, despite identical parameter settings. This variability may lead to incomplete data at the perimeter, thus potentially making volume-wide measurements unreliable.

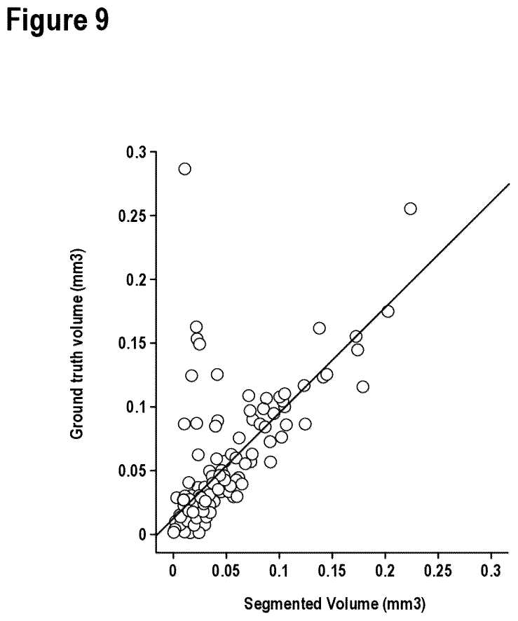

Validation. Model performance for HRF and IHRM was evaluated against the annotations on the validation set using DICE, measuring the overlap between annotations and model predictions. Median and average DICE scores on the validation set were 71% and 65%, respectively, which are considered to be good scores, given the variability of DICE for small-size objects, such as HRF. Median and average ground-truth volumes on the B-scan level were also compared with the segmented volumes. These were 30 and 50 nL for the ground-truth volumes, respectively, and 20 and 40 nL for the segmented volumes, respectively. A scatter plot comparing ground-truth and segmented volumes is shown in FIG. 9.

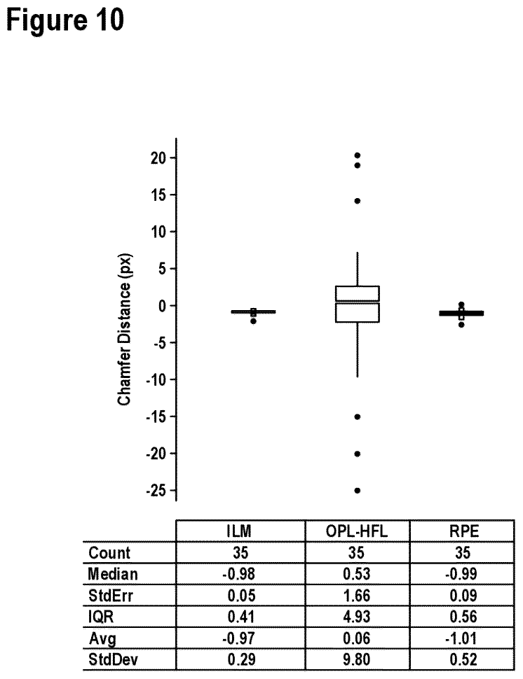

Performance for layers was evaluated using Chamfer distance, 47 which measures the average distance (in pixels) between the annotated and predicted elevation of layers across A-Scans. The median (standard error) Chamfer distances across B-scans in the validation set for ILM, OPL-HFL, and RPE were −0.98 (0.05), 0.53 (1.66), and −1.0 (0.08) pixels, respectively, which are below the optical resolution of SD-OCT machines. Boxplots are shown in FIG. 10.

### Statistical Analysis

Prior to feature extraction and statistical analysis, HRF had been pre-specified as a biomarker of interest, potentially representing chronic inflammation. Mean values and treatment-group comparisons were estimated for each HRF parameter separately using a Mixed Model for Repeated Measures (MMRM), adjusted for baseline HRF value, treatment arm, visit, visit-by-treatment-arm interaction, and baseline BCVA, as well as the randomization-stratification factors: Baseline BCVA category (<64 letters vs>=64 letters); prior intravitreal anti-VEGF therapy (yes or no); and region (United States and Canada, Asia, and rest of the world). An unstructured covariance structure was assumed. Given the positively-skewed distribution of the data, sensitivity analyses were conducted using a stratified Wilcoxon rank-sum test. No adjustment to the significance level was made to account for multiple treatment comparisons or analyses at multiple time points, therefore p-values should be interpreted in an exploratory context.

### Results

At baseline, unadjusted mean HRF volumes were comparable for all three treatment arms, and HRF were present in at least 75% of patients in each arm, diameter, and location (Table 2). Baseline characteristics for the patients included in this analysis were well balanced across all three treatment arms (Table 3).

TABLE 2
HRF Volume and Presence at Baseline

| HRF Volume | | Faricimab 6.0 mg | | Faricimab 6.0 mg | | Aflibercept 2.0 mg | |
|---|---|---|---|---|---|---|---|
| [pL] | | Q8W | | T&E | | Q8W | |
| Inner Retina, | Mean (SE) | 222.4 | (11.9) | 240.2 | (14.5) | 239.0 | (14.0) |
| 1 mm | HRF Present | 459/512 | (89.6%) | 472/519 | (90.9%) | 438/496 | (88.3%) |
| Inner Retina, | Mean (SE) | 1170.7 | (51.6) | 1310.2 | (61.5) | 1428.3 | (70.0) |
| 3 mm | HRF Present | 509/512 | (99.4%) | 517/519 | (99.6%) | 494/496 | (99.6%) |
| Outer Retina, | Mean (SE) | 162.5 | (11.8) | 185.7 | (14.0) | 202.0 | (14.7) |
| 1 mm | HRF Present | 402/512 | (78.5%) | 398/519 | (76.7%) | 389/496 | (78.4%) |
| Outer Retina, | Mean (SE) | 1494.8 | (84.9) | 1656.5 | (97.6) | 1823.6 | (105.6) |
| 3 mm | HRF Present | 500/512 | (97.7%) | 506/519 | (97.5%) | 483/496 | (97.4%) |

Measured, unadjusted mean values are presented for baseline HRF volume. pL: picoliters; SE: standard error of the mean; 1 mm: 1-mm diameter ETDRS ring; 3 mm: 3-mm diameter ETDRS ring.

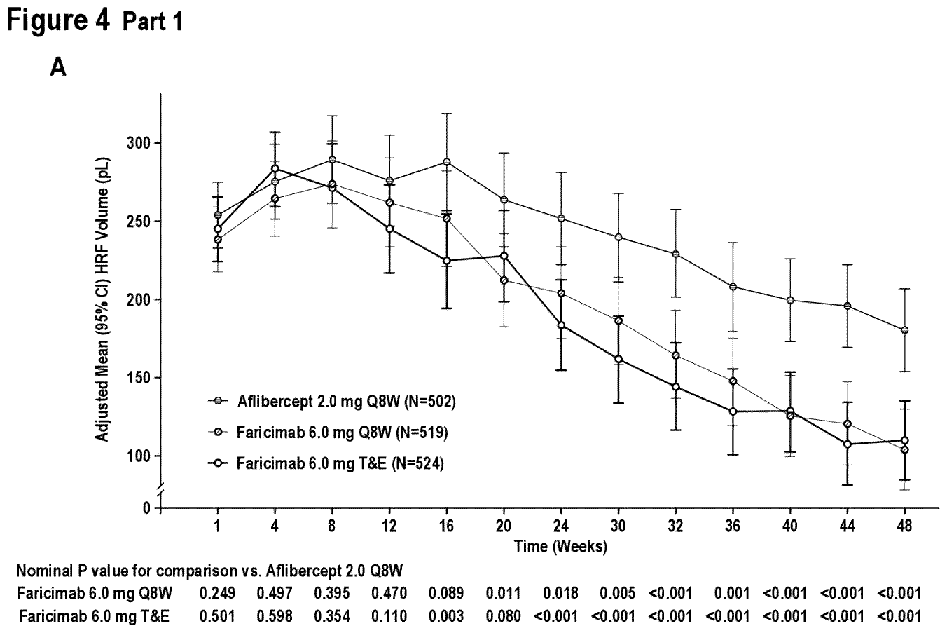

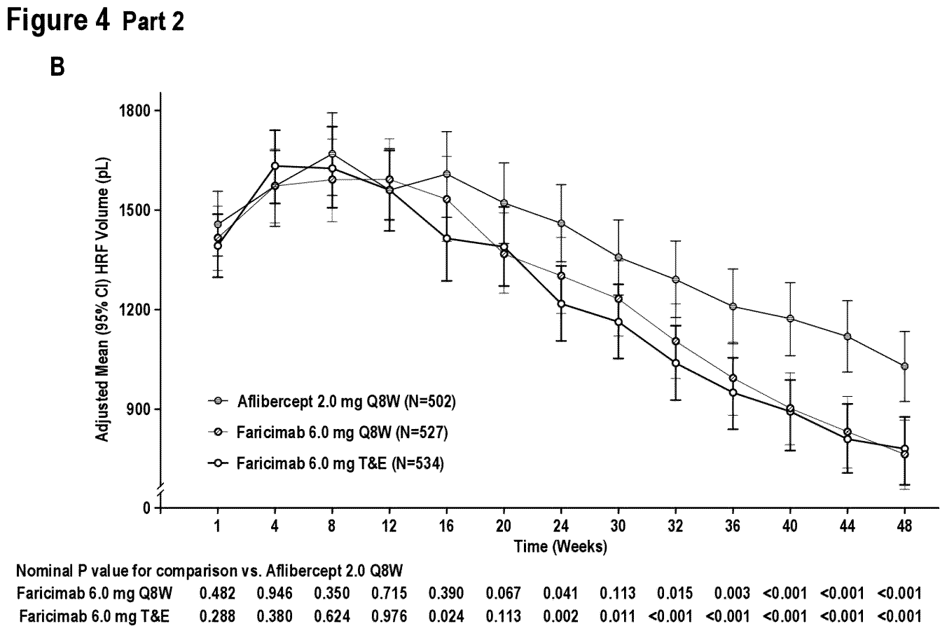

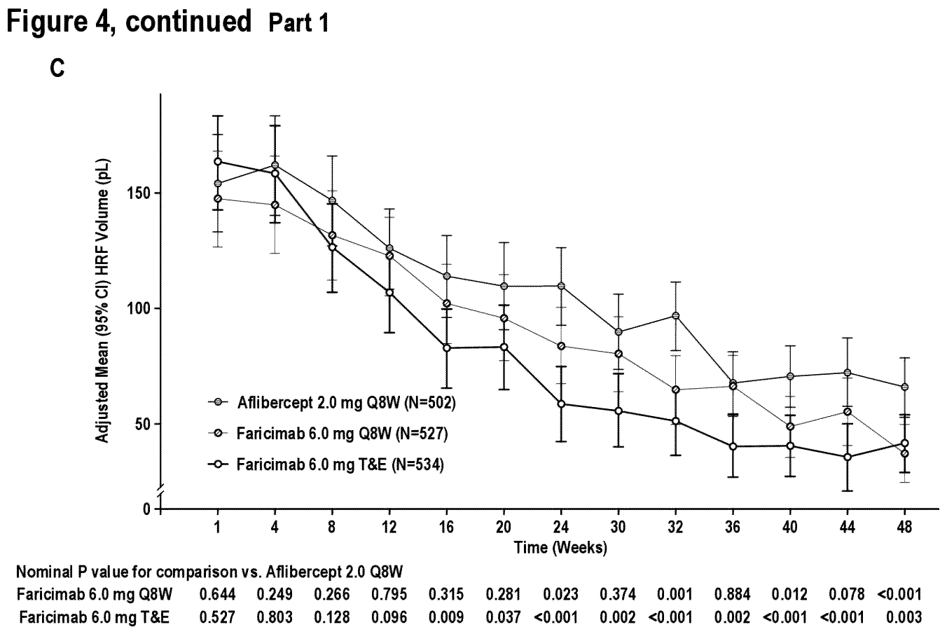

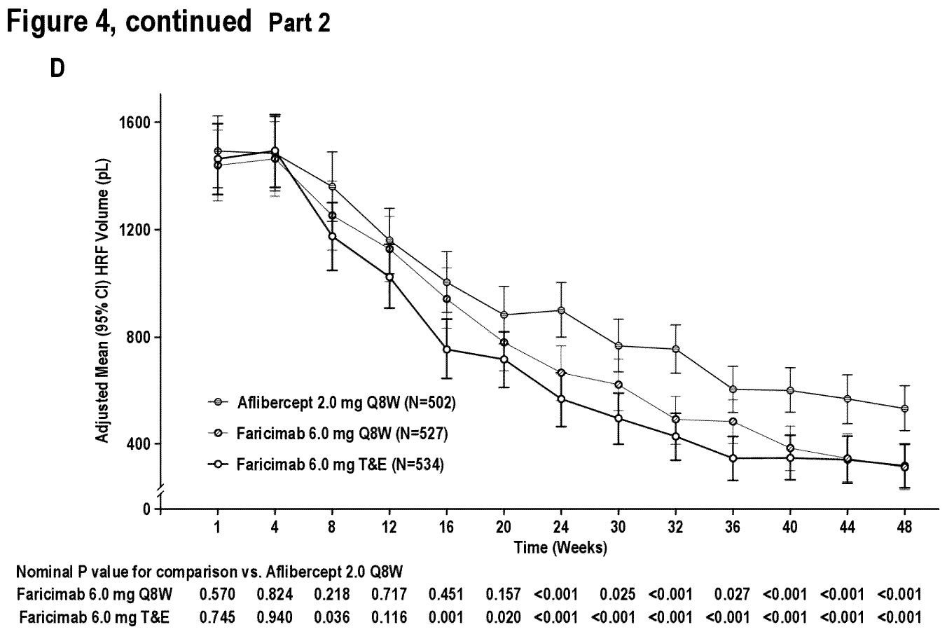

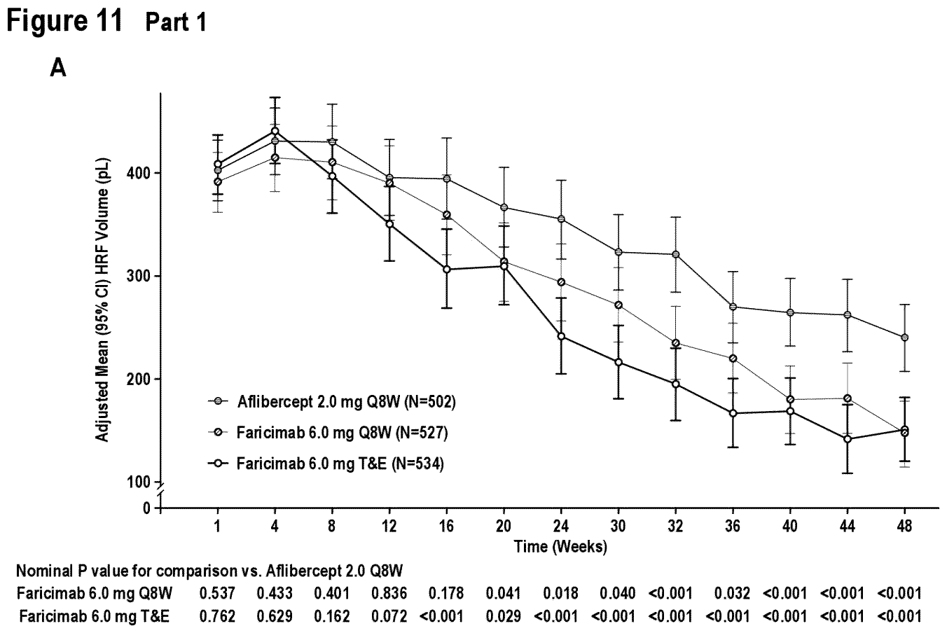

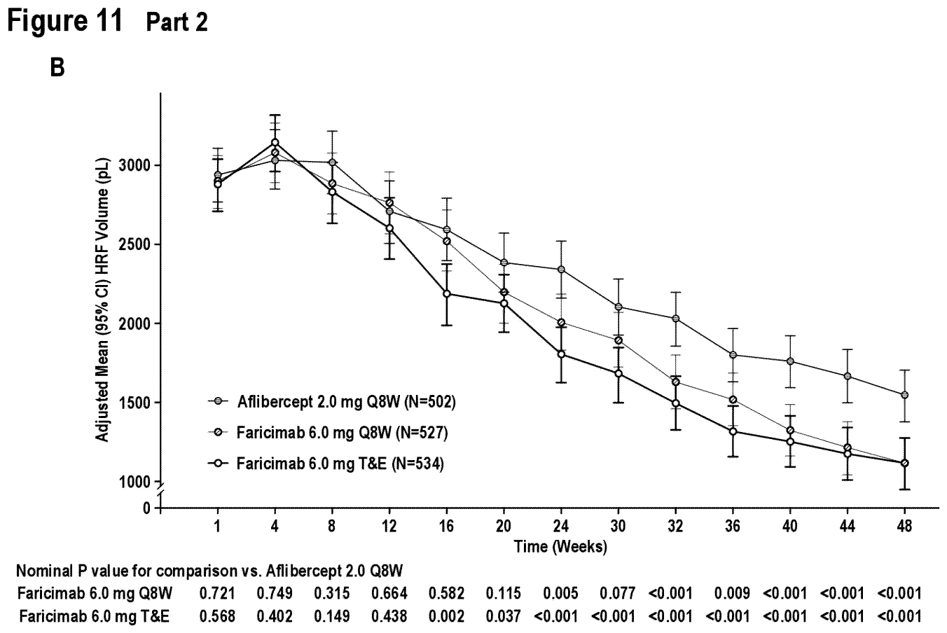

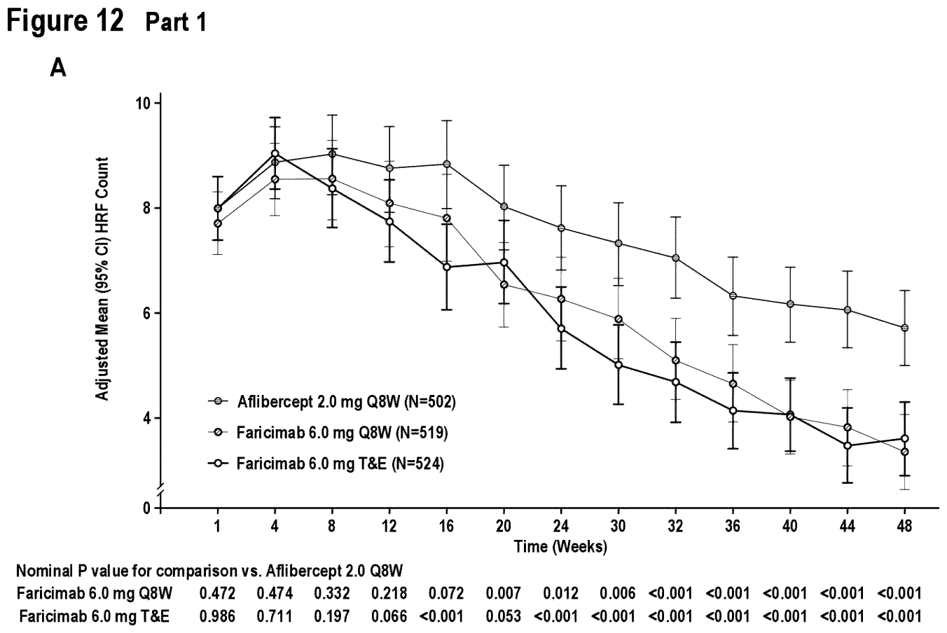

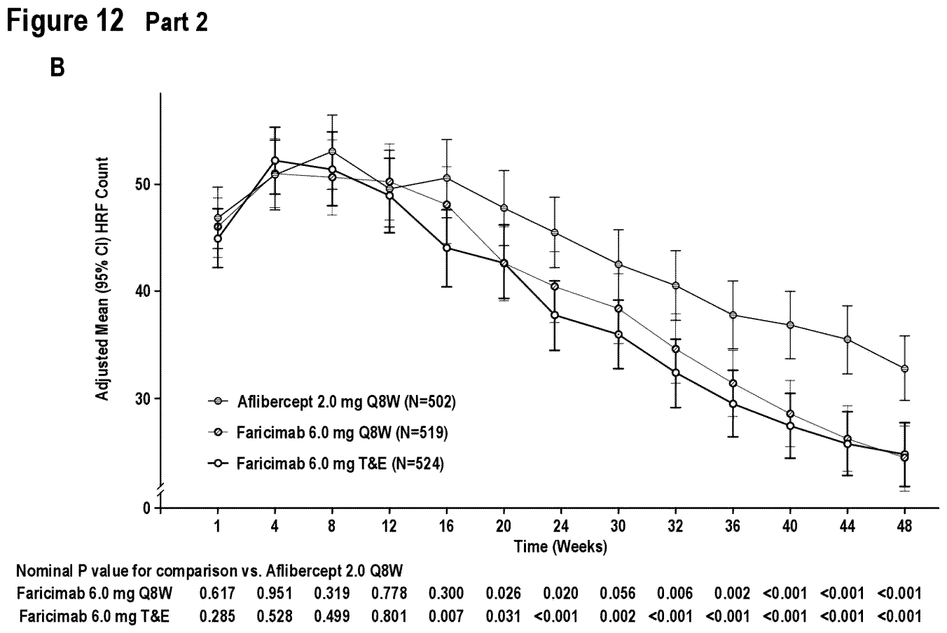

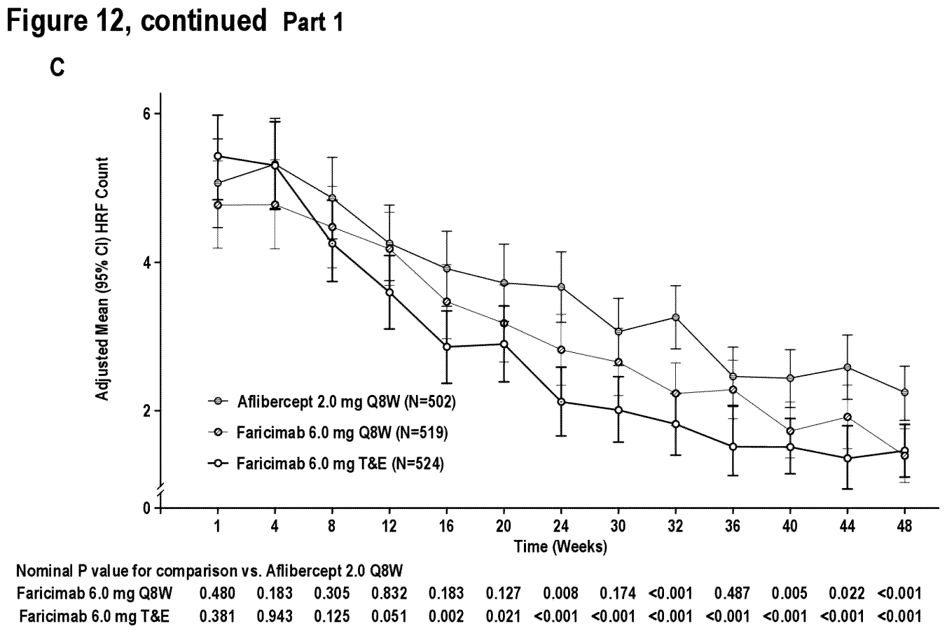

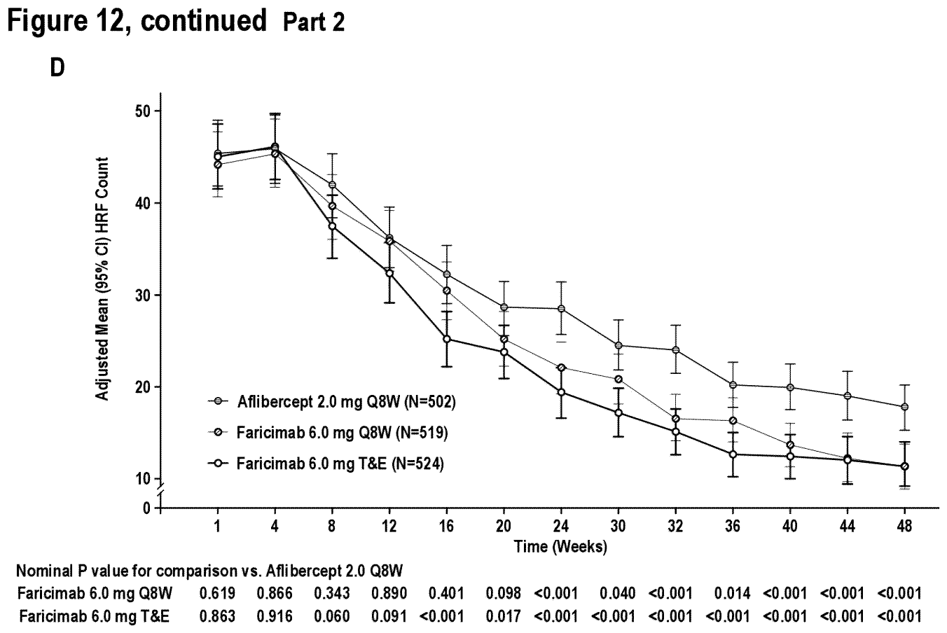

Adjusted-mean HRF volumes for the inner and outer retina within the 1- and 3-mm diameter are shown in FIG. 4, and reported in Table 3 for baseline, and weeks 8, 16, and 48. In both the inner and outer retina, and at both diameters, treatment with faricimab (Q8W and T&E) led to a greater reduction in HRF volume compared to aflibercept (nominal p<0.001). Volumetric HRF differences remained nominally significant when assessed in the total retina (inner and outer retina combined; FIG. 11). To supplement the volumetric analyses, HRF counts were also analyzed and comparable reductions were measured (FIG. 12).

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

TABLE 3
HRF Volume at Baseline, Week 8, Week 16 and Week 48

| HRF Volume | | | Farichmuab 6.0 mg | Faricimab 6.0 mg | Aflibercept 2.0 mg |
|---|---|---|---|---|---|
| [pL] | | | Q8W | T&E | Q8W |
| Inner Retina, 1 mm | Bassline | n | [symbol] 12 | [symbol] 18 | 4 [symbol] |

| | | | | | | |
|---|---|---|---|---|---|---|
| Mean (SE) | [symbol] 22.4 | (11.9) | 240.2 | (14. [symbol] ) | 23 [symbol] . [symbol] | (1 [symbol] .0) |
| Median (IQR) | 138.2 | (40. [symbol] , 306. [symbol] ) | 121.8 | (39.4, [symbol] 0 [symbol] .1) | 118.4 | ( [symbol] 8.7, 319.1) |

| | | | | |
|---|---|---|---|---|
| Week 8 | n | [symbol] | [symbol] 0 | [symbol] 3 [symbol] |

| | | | | | | |
|---|---|---|---|---|---|---|
| Adjusted Mean (SE) | 273. [symbol] | (14.0) | 272.3 | (1 [symbol] . [symbol] ) | 28 [symbol] .3 | (1 [symbol] .2) |
| Median (IQR) | 173.2 | ( [symbol] .9, 3 [symbol] .5) | 146.2 | (39.0, 360.4) | 177.7 | (4 [symbol] .7, 3 [symbol] .2) |

| | | | | |
|---|---|---|---|---|
| Week 16 | n | 470 | 488 | 4 [symbol] 7 |

| | | | | | | |
|---|---|---|---|---|---|---|
| Adjusted Mean (SE) | 2 [symbol] 1.7 | (1 [symbol] .7) | 224.7 | (1 [symbol] . [symbol] ) | 287. [symbol] | (1 [symbol] . [symbol] ) |
| Median (IQR) | 138. [symbol] | (3 [symbol] .7, 328.6) | [symbol] .8 | (2 [symbol] . [symbol] , 302. [symbol] ) | 1 [symbol] .2 | (3 [symbol] . [symbol] , 3 [symbol] . [symbol] ) |

| | | | | |
|---|---|---|---|---|
| Week 48 | n | 414 | 44 [symbol] | 42 [symbol] |

| | | | | | | |
|---|---|---|---|---|---|---|
| Adjusted Mean (SE) | 104.1 | (13.2) | 110. [symbol] , | (22.0) | 180.3 | (13.3) |
| Median (IQR) | 27. [symbol] | ( [symbol] .10 [symbol] .9) | 36.1 | ( [symbol] .0, 121.4) | [symbol] . [symbol] | (0.0, 23 [symbol] .0) |

| | | | | | |
|---|---|---|---|---|---|
| Across All Visits | p-value (MMRM) [symbol] | 0.0006 | <0.0001 | 85.6 | (0.0, 235.0) |
| | p-value ( [symbol] ) [symbol] | <0.0001 | <0.0001 | | |

| | | | | | |
|---|---|---|---|---|---|
| Inner Retina, 3 mm | Bassline | n | [symbol] 12 | [symbol] 19 | [symbol] 6 |

| | | | | | | |
|---|---|---|---|---|---|---|
| Mean (SE) | 1170.7 | ( [symbol] 1.6) | 1310.2 | ( [symbol] . [symbol] ) | 1428.3 | (70.0) |
| Median (IQR) | 784.2 | (320.2, 1 [symbol] 17.8) | 844.6 | (379. [symbol] , 17 [symbol] 2. [symbol] ) | [symbol] 1. [symbol] | ( [symbol] . [symbol] , 2013. [symbol] ) |

| | | | | |
|---|---|---|---|---|
| Week 8 | n | [symbol] | 4 [symbol] 0 | 439 |

| | | | | | | |
|---|---|---|---|---|---|---|
| Adjusted Mean (SE) | 1 [symbol] 91.0 | (62. [symbol] ) | 1 [symbol] 8.3 | (62.2) | 1 [symbol] .2 | ( [symbol] 3.7) |
| Median (IQR) | 10 [symbol] 8.0 | (467.3, 2087. [symbol] ) | 11 [symbol] .0 | (388.9, 23 [symbol] .1) | 12 [symbol] 1.0 | ( [symbol] 78. [symbol] , [symbol] . [symbol] ) |

| | | | | |
|---|---|---|---|---|
| Week 16 | n | 377 | [symbol] 89 | 467 |

| | | | | | | |
|---|---|---|---|---|---|---|
| Adjusted Mean (SE) | 1 [symbol] .3 | ( [symbol] 4. [symbol] ) | 1414.1 | (63.9) | [symbol] 08. [symbol] | ( [symbol] . [symbol] ) |
| Median (IQR) | 1010. [symbol] | (3 [symbol] 8.2, 1 [symbol] 1.3) | [symbol] 8 [symbol] . [symbol] | ( [symbol] 23.1, 2028.8) | 1052.7 | (417. [symbol] , 2 [symbol] . [symbol] ) |

| | | | | |
|---|---|---|---|---|
| Week 48 | n | 414 | 44 [symbol] | 421 |

| | | | | | | |
|---|---|---|---|---|---|---|
| Adjusted Mean (SE) | 763.9 | ( [symbol] 3.1) | 777.2 | ( [symbol] .2) | 103 [symbol] .6 | ( [symbol] 3.7) |
| Median (IQR) | 35 [symbol] .8 | (13 [symbol] .880.3) | 3 [symbol] . [symbol] | (135. [symbol] , 1028.0) | 616.4 | (21 [symbol] .8, [symbol] .1) |

| | | | |
|---|---|---|---|
| Across All Visits | p-value (MMRM) [symbol] | 0.0142 | 0.0034 |
| | p-value ( [symbol] ) [symbol] | <0.0001 | <0.0001 |

| | | | | | |
|---|---|---|---|---|---|
| Outer Retina, 1 mm | Bassline | n | 512 | 51 [symbol] | [symbol] |

| | | | | | | |
|---|---|---|---|---|---|---|
| Mean (SE) | 102.5 | (11.8) | 185.7 | ( [symbol] 4.0) | 202.0 | (14.7) |
| Median (IQR) | 63.3 | ( [symbol] .0, 18 [symbol] .4) | 63.1 | ( [symbol] .1, 213.8) | 75.7 | ( [symbol] . [symbol] , 234.1) |

| | | | | |
|---|---|---|---|---|
| Week 8 | n | [symbol] | 4 [symbol] 0 | 439 |

| | | | | | | |
|---|---|---|---|---|---|---|
| Adjusted Mean (SE) | 131.9 | (9.8) | 12 [symbol] . [symbol] | ( [symbol] .8) | 146. [symbol] | (10.0) |
| Median (IQR) | 44. [symbol] | ( [symbol] .0, 1 [symbol] 0. [symbol] ) | 43. [symbol] | (0.0, 162.1) | 49. [symbol] | (4.7, 17 [symbol] . [symbol] ) |

| | | | | |
|---|---|---|---|---|
| Week 16 | n | 47 [symbol] | 488 | 467 |

| | | | | | | |
|---|---|---|---|---|---|---|
| Adjusted Mean (SE) | 102.1 | (8.8) | 83.0 | ( [symbol] .7) | 114. [symbol] | ( [symbol] .9) |
| Median (IQR) | 26.2 | ( [symbol] .0, 11 [symbol] . [symbol] ) | 2 [symbol] .9 | (0.0, 106.4) | 28.7 | (0.0, 13 [symbol] .4) |

| | | | | |
|---|---|---|---|---|
| Week 48 | n | 414 | 449 | 421 |

| | | | | | | |
|---|---|---|---|---|---|---|
| Adjusted Mean (SE) | 37.2 | ( [symbol] .4) | 41.4 | ( [symbol] .3) | [symbol] .7 | (6. [symbol] ) |
| Median (IQR) | 0.0 | (0.0, 33.1) | 0.0 | (0.0, [symbol] .1) | 8. [symbol] | (0.0, 68. [symbol] ) |

| | | | |
|---|---|---|---|
| Across All Visits | p-value (MMRM) [symbol] | 0.03 [symbol] 0 | 0.000 [symbol] |
| | p-value ( [symbol] ) [symbol] | <0.0001 | <0.0001 |

| | | | | | |
|---|---|---|---|---|---|
| Outer Retina, 3 mm | Bassline | n | [symbol] 12 | [symbol] 19 | [symbol] |

| | | | | | | |
|---|---|---|---|---|---|---|
| Mean (SE) | 149 [symbol] .8 | (84.9) | 1 [symbol] . [symbol] | ( [symbol] 7. [symbol] ) | 182 [symbol] . [symbol] | (105.0) |
| Median (IQR) | 742.2 | (238.4, 19 [symbol] 1.7) | 780. [symbol] | (209.1, 2118. [symbol] ) | 847.4 | (2 [symbol] . [symbol] , 2 [symbol] 1.8) |

| | | | | |
|---|---|---|---|---|
| Week 8 | n | [symbol] | [symbol] | 439 |

| | | | | | | |
|---|---|---|---|---|---|---|
| Adjusted Mean (SE) | 12 [symbol] .3 | (64.7) | 117 [symbol] .2 | (64.2) | 1 [symbol] 1.0 | ( [symbol] .8) |
| Median (IQR) | [symbol] .8 | (172.8, 1529. [symbol] ) | [symbol] 7 [symbol] .0 | (133. [symbol] , 1 [symbol] .8) | [symbol] 8 [symbol] .9 | (177. [symbol] , 1 [symbol] 48.4) |

| | | | | |
|---|---|---|---|---|
| Week 16 | n | 477 | 48 [symbol] | 4 [symbol] 7 |

| | | | | | | |
|---|---|---|---|---|---|---|
| Adjusted Mean (SE) | 945.8 | (5 [symbol] .8) | 7 [symbol] 4. [symbol] | ( [symbol] .3) | 1003.4 | ( [symbol] 7.7) |
| Median (IQR) | 32 [symbol] .9 | (89.9, 10 [symbol] .4) | 32 [symbol] . [symbol] | ( [symbol] 0. [symbol] , [symbol] 7 [symbol] . [symbol] ) | 4 [symbol] .8 | (11 [symbol] . [symbol] , 145 [symbol] . [symbol] ) |

| | | | | |
|---|---|---|---|---|
| Week 48 | n | 414 | [symbol] | 421 |

| | | | | | | |
|---|---|---|---|---|---|---|
| Adjusted Mean (SE) | 311.7 | (42. [symbol] ) | 319. [symbol] | (41. [symbol] ) | [symbol] 33. [symbol] | (4 [symbol] .0) |
| Median (IQR) | 8 [symbol] . [symbol] | (2 [symbol] .1, 273. [symbol] ) | 107. [symbol] | ( [symbol] 3.0, 2 [symbol] 2. [symbol] ) | 188. [symbol] | ( [symbol] 7.7, 60 [symbol] .3) |

| | | | |
|---|---|---|---|
| Across All Visits | p-value (MMRM) [symbol] | 0.00 [symbol] 2 | <0.0001 |
| | p-value ( [symbol] ) [symbol] | <0.0001 | <0.0001 |

| | | | | | |
|---|---|---|---|---|---|
| Total Retina, 1 mm | Bassline | n | [symbol] 12 | [symbol] 1 [symbol] | [symbol] |

| | | | | | | |
|---|---|---|---|---|---|---|
| Mean (SE) | 384. [symbol] | (19.0) | 4. [symbol] 8 | (23.1) | 440.9 | (23.0) |
| Median (IQR) | 249.6 | (10 [symbol] .9, 5 [symbol] 01. [symbol] ) | [symbol] . [symbol] | (86.2, [symbol] 78.8) | 2 [symbol] . [symbol] | (201.4, [symbol] 04.7) |

| | | | | |
|---|---|---|---|---|
| Week 8 | n | [symbol] | 4 [symbol] 0 | 4 [symbol] |

| | | | | | | |
|---|---|---|---|---|---|---|
| Adjusted Mean (SE) | 410.0 | (18.1) | 39 [symbol] . [symbol] | (18.0) | 43 [symbol] .3 | (18. [symbol] ) |
| Median (IQR) | 247.7 | (89.1, [symbol] 38.4) | 2. [symbol] 2 | ( [symbol] 1. [symbol] , [symbol] 7.2) | 27 [symbol] . [symbol] | ( [symbol] 3.2, 607.1) |

| | | | | |
|---|---|---|---|---|
| Week 16 | n | 47 [symbol] | 488 | 467 |

| | | | | | | |
|---|---|---|---|---|---|---|
| Adjusted Mean (SE) | [symbol] .0 | (19. [symbol] ) | 306.8 | (1 [symbol] . [symbol] ) | 3 [symbol] .7 | (19. [symbol] ) |
| Median (IQR) | 1 [symbol] 0.8 | (4 [symbol] . [symbol] , 4 [symbol] 9.4) | 149.6 | (39.3, 4 [symbol] 1. [symbol] ) | 2 [symbol] .7 | (61.9, [symbol] 31.4) |

| | | | | |
|---|---|---|---|---|
| Week 48 | n | 414 | 4 [symbol] | 421 |

| | | | | | | |
|---|---|---|---|---|---|---|
| Adjusted Mean (SE) | 14 [symbol] . [symbol] | (10.1) | 1 [symbol] 0. [symbol] | (1 [symbol] .8) | 240.0 | (1 [symbol] .2) |
| Median (IQR) | 1 [symbol] .1 | ( [symbol] .0, 141.3) | [symbol] 9.9 | ( [symbol] .1, 172. [symbol] ) | 100.4 | (1 [symbol] .4, [symbol] 31.4) |

| | | | |
|---|---|---|---|
| Across All Visits | p-value (MMRM) [symbol] | 0.00 [symbol] | <0.0001 |
| | p-value ( [symbol] ) [symbol] | <0.0001 | <0.0001 |

| | | | | | |
|---|---|---|---|---|---|
| Total Retina, 3 mm | Bassline | n | 512 | [symbol] 1 [symbol] | 49 [symbol] |

| | | | | | | |
|---|---|---|---|---|---|---|
| Mean (SE) | 266 [symbol] . [symbol] | (114.7) | 2 [symbol] 86.7 | (1 [symbol] 6. [symbol] ) | 3251.9 | (1 [symbol] 8.1) |
| Median (IQR) | 1876.7 | (77 [symbol] .0, [symbol] 7 [symbol] . [symbol] ) | 1 [symbol] 1 [symbol] .2 | (7 [symbol] . [symbol] , 3877. [symbol] ) | 2111. [symbol] | (804.0, 4718. [symbol] ) |

| | | | | |
|---|---|---|---|---|
| Week 8 | n | [symbol] | 460 | 439 |

| | | | | | | |
|---|---|---|---|---|---|---|
| Adjusted Mean (SE) | 2880. [symbol] | (97.8) | 28 [symbol] 4.0 | ( [symbol] 7.1) | 3012.8 | ( [symbol] 2. [symbol] ) |
| Median (IQR) | 18 [symbol] 1.6 | (778.3, 3 [symbol] 76. [symbol] ) | 1 [symbol] . [symbol] | (625.1, 3 [symbol] 7. [symbol] ) | 2103.8 | (862.1, 4475. [symbol] ) |

| | | | | |
|---|---|---|---|---|
| Week 16 | n | 477 | 48 [symbol] | 467 |

| | | | | | | |
|---|---|---|---|---|---|---|
| Adjusted Mean (SE) | 2 [symbol] 19.4 | (98.1) | 21 [symbol] 4.3 | (97.2) | 2 [symbol] 1. [symbol] | ( [symbol] . [symbol] ) |
| Median (IQR) | 1480.7 | ( [symbol] 76.6, 3174.3) | 147 [symbol] .1 | (517.8, 3333.3) | 1819.2 | ( [symbol] 06.7, 40 [symbol] 8.4) |

| | | | | |
|---|---|---|---|---|
| Week 48 | n | 414 | [symbol] | 421 |

| | | | | | | |
|---|---|---|---|---|---|---|
| Adjusted Mean (SE) | 1112.3 | (81.8) | 1118.6 | (80. [symbol] ) | 1 [symbol] 3.6 | (83. [symbol] ) |
| Median (IQR) | [symbol] 4.2 | (184.8, 1179.7) | [symbol] .8 | (217.1, 1 [symbol] 12. [symbol] ) | 82 [symbol] .0 | (32 [symbol] .2, 2488.2) |

| | | | |
|---|---|---|---|
| Across All Visits | p-value (MMRM) [symbol] | 0.0112 | 0.0001 |
| | p-value ( [symbol] ) [symbol] | <0.0001 | <0.0001 |

[symbol]

Comparison to aflibercept and All p-values are nominal [symbol]
indicates data missing or illegible when filed

Following treatment initiation, an initial rise in adjustment-mean HRF volume was observed in the inner retina for all three treatment arms, peaking around week 8 (FIGS. 4a and 4b). Conversely, no such rise was observed in the outer retina, regardless of diameter assessed (FIGS. 4c and 4d). Table 4 shows the baseline (unadjusted) and week 8 (adjusted) mean HRF volumes and the factor by which the HRF volumes increased from baseline to week 8.

TABLE 4
HRF Volumes in The Inner Retina at Select Visits

| | | | Faricimab | | Faricimab | | Aflibercept | |
|---|---|---|---|---|---|---|---|---|
| HRF Volume | | | 6.0 mg Q8W | | 6.0 mg T&E | | 2.0 mg Q8W | |
| [pL] | | | (N = 519) | | (N = 524) | | (N = 502) | |
| Inner Retina, | Baseline | Mean (SE) | 222.4 | (11.9) | 240.2 | (14.5) | 239.0 | (14.0) |
| 1 mm | Week 8 | Adjusted Mean (SE) | 273.5 | (14.0) | 272.1 | (13.9) | 289.3 | (14.2) |
| | | Factor (Week | 1.23 | | 1.13 | | 1.21 | |
| | | 8/Baseline) | | | | | | |
| Inner Retina, | Baseline | Mean (SE) | 1170.7 | (51.6) | 1310.2 | (61.5) | 1428.3 | (70.0) |
| 3 mm | Week 8 | Adjusted Mean (SE) | 1591.0 | (62.6) | 1628.3 | (62.2) | 1669.2 | (63.7) |

| | | | |
|---|---|---|---|
| Factor (Week | 1.36 | 1.24 | 1.17 |
| 8/Baseline) | | | |

pL: picoliters; SE: standard error of the mean; 1 mm: 1-mm diameter ETDRS ring; 3 mm: 3-mm diameter ETDRS ring.

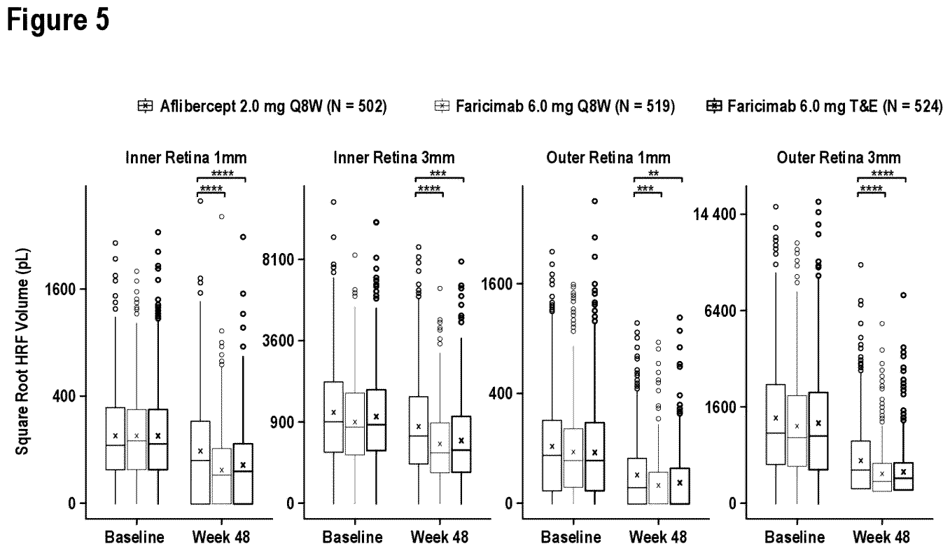

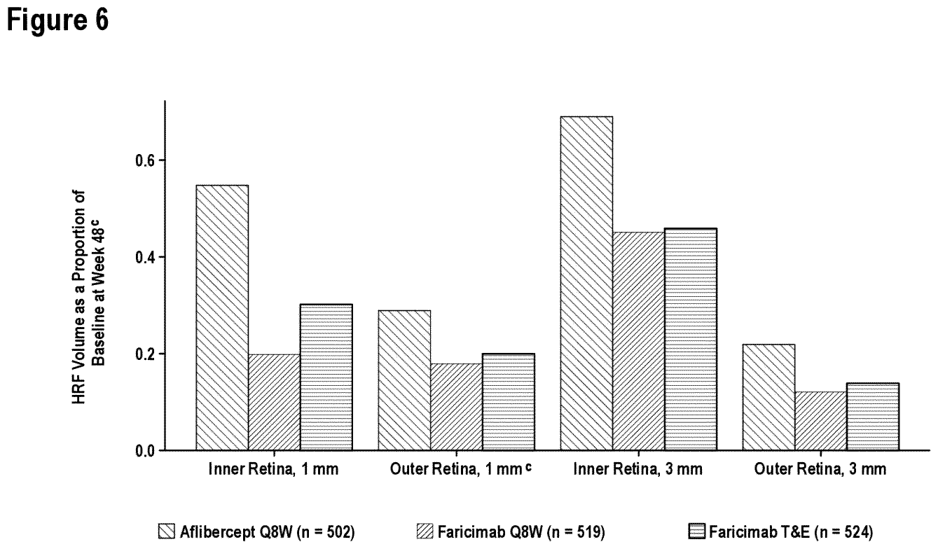

Reduction in HRF volume with aflibercept was predominantly observed in the outer retina, whereas reductions in HRF volume occurred in both the inner and outer retina in faricimab-treated eyes (FIG. 5).

In DME, HRF represent a potential biomarker for disease severity and progression. Some DME patients with a high HRF burden benefit from switching therapies from anti-VEGF to steroid, which may suggest that this patient population benefits from treatment with an additional mode of action. The phase 3 clinical trials YOSEMITE and RHINE have shown that simultaneous inhibition of Ang-2 and VEGF-A with faricimab maintains vision gains and controls anatomical outcomes with extended durability. The aim of this analysis was to assess whether combined Ang-2 and VEGF-A suppression with faricimab leads to a greater reduction in retinal HRF compared to aflibercept. The large data set, the fully automated detection and volumetric quantification of HRF, and the objective nature of the algorithmic approach make this a robust analysis of a potentially key biomarker in DME.

To the best of our knowledge, this is the first volumetric assessment of HRF in DME. Analyzing the substantial SD-OCT dataset from the phase 3 YOSEMITE and RHINE clinical trials, it was shown that HRF volumes decrease more with faricimab (regardless of regimen: Q8W or T&E) than with aflibercept. The difference was noticeable as early as week 16 and maintained up to week 48 (the last study visit assessed in the current analysis). The effect was seen in both the central 1-mm and 3-mm diameters of the retina, as well as in both the inner and outer retina. It was observed that treatment with aflibercept caused reductions in HRF volume in the outer retina, with minimal changes in the inner retina. By contrast, treatment with faricimab resulted in HRF volume reductions in all retinal layers.

In the immediate period following treatment initiation, it was observed that HRF volumes increased in the inner retina (FIGS. 4a and 4b). One explanation for this finding is that at baseline, the presence of diffuse edema and turbid cysts may mask some HRF, thus preventing detection by the algorithm. Turbid fluid in the retina is not uncommon in DME, loculating as foci of varying contrast, which in turn likely masks the identification of small bright objects such as HRF. In all three study arms of YOSEMITE and RHINE, central subfield thickness reduced rapidly in the first 8-12 weeks, which aligns well with the inflection point at which HRF volumes begin to decrease (FIGS. 4a and 4b). At this stage, the reduction in fluid is likely sufficient to allow for more accurate quantification of HRF, thus potentially causing an artificial rise in HRF volume prior to the expected reduction. No such initial rise in the curves was seen in the outer retina (FIGS. 4c and 4d), which aligns with the fact that IRF largely affects the inner retina.

Prior to treatment initiation at the baseline visit, more than three-quarters of study eyes (76.7% to 99.6% across all arms, depending on ETDRS ring and location in inner vs outer retina) had HRF present. This is in accordance with previous reports on the prevalence of HRF, where approximately 90% of eyes with DME were shown to exhibit this feature on SD-OCT. HRF volume and count decreased concurrently, showing that these foci did not simply get smaller, but were also reducing in number, indicating resolution. Since DME is a disease of the intraretinal vessels, which are predominantly located in the inner retina, this outcome suggests that faricimab has an additional effect on disease severity and progression.

There are two main hypotheses on the morphological correlate of HRF detected by SD-OCT. The first proposes that HRF in DME are activated microglia or infiltrated leukocytes, thus representing a retinal inflammatory response. The second hypothesis suggests that HRF are protein and/or lipid exudates resulting from the breakdown of the blood-retinal barrier. As these hypotheses are not mutually exclusive, some groups have also discussed the potential of both hypotheses applying concurrently, with smaller HRF (≤30 μm) proposed to correspond to inflammatory cells and larger objects (>30 μm) representing macromolecular exudates. Histological studies on HRF are currently still lacking in DME, and are necessary to confirm these hypotheses.

Several studies have shown that intraocular, free VEGF is not detectable in biological samples for an average of 34 days (ranibizumab) and 67 days (aflibercept) after intravitreal administration, showing that near-total VEGF suppression is achieved with standard, clinically used doses. Furthermore, analysis of aqueous humour samples collected during the YOSEMITE and RHINE trials has shown that aflibercept does not reduce Ang-2 levels in patients. Without being bound by a theory, it is believed that the greater HRF reduction seen with faricimab treatment compared to aflibercept cannot be attributed solely to its anti-VEGF properties.

In a recently published preclinical study investigating retinal inflammation in JR5558 mice, dual Ang-2/VEGF-A inhibition led to a significantly greater reduction in Ibal⁺ immune cells (including macrophages and microglia) than VEGF-A or Ang-2 inhibition alone (Canonica J, et al. Front Cell Neurosci. 2023; 17:1192464). This effect was maintained at 5 weeks after treatment for dual Ang-2/VEGF-A and Ang-2 inhibition, but not with VEGF-A inhibition alone, suggesting that Ang-2 inhibition may be driving the anti-inflammatory effect seen with dual Ang-2/VEGF-A inhibition. Using the retinal ischemia-reperfusion injury model, the authors were also able to demonstrate that dual Ang-2/VEGF-A inhibition was significantly better at preventing retinal vascular leakage compared to blocking either Ang-2 or VEGF-A alone. Thus, without being bound by a theory, it is believed the additional impact seen with faricimab on HRF volume reduction is likely at least in part attributable to faricimab's anti-Ang-2 effect.

In summary, it was shown that faricimab treatment results in greater resolution of HRF than aflibercept; this difference is established at the end of the matched loading phase (week 16). The relevance of this effect of faricimab requires more investigation, not only to further establish its clinical relevance, but also to characterize the dynamics of HRF change in the longer term. Further evaluation of HRF volumes and anatomical parameters is needed in the year 2 data of YOSEMITE and RHINE to assess clinical significance.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method of reducing hyperreflective foci (HRF) in an eye of a patient suffering from Diabetic Macular Edema (DME), the method comprising:
administering to the patient an effective amount of a bispecific antibody which binds to human vascular endothelial growth factor (VEGF) and to human angiopoietin-2 (ANG-2), and comprises the amino acid sequences of SEQ ID NO: 17, of SEQ ID NO: 18, of SEQ ID NO: 19, and of SEQ ID NO: 20.

2. The method of claim 1, wherein the effective amount of the bispecific antibody is sufficient to reduce HRF volume and/or count after 48 weeks of treatment.

3. The method of claim 2, wherein the HRF volume after 48 weeks of treatment is less than 0.5 relative to the HRF volume prior to treatment.

4. The method of claim 2, wherein the HRF volume is reduced in the central 1-mm diameter of the retina and/or in the central 3-mm diameter of the retina.

5. A method of treating a patient suffering from Diabetic Macular Edema (DME), the method comprising:
administering to the patient an effective amount of a bispecific antibody which binds to human vascular endothelial growth factor (VEGF) and to human angiopoietin-2 (ANG-2), and comprises the amino acid sequences of SEQ ID NO: 17, of SEQ ID NO: 18, of SEQ ID NO: 19, and of SEQ ID NO: 20;
measuring hyperreflective foci (HRF) in an eye of the patient after 16 and/or 48 weeks of treatment; and
adjusting administration dosing interval based on the HRF volume and/or count.

6. The method of claim 5, wherein the dosing interval is shortened if HRF volume and/or count is not reduced relative to the HRF volume and/or count prior to treatment.

7. The method of claim 5, wherein the dosing interval is extended if HRF volume and/or count is reduced relative to the HRF volume and/or count prior to treatment.

8. The method of claim 5, wherein the dosing interval is extended if HRF volume after 48 weeks of treatment is less than 0.5 relative to the HRF volume prior to treatment.

9. The method of claim 5, wherein the HRF volume is measured in the central 1-mm diameter of the retina or in the central 3-mm diameter of the retina.

10. The method of claim 5, wherein reducing HRF prolongs the time to retreatment and/or prolongs the time to loss of visual acuity (e.g., reduces the progression and/or severity of the disease).

11. The method of claim 5, wherein the patient has vision loss due to center-involving DME.

12. The method of claim 5, wherein the bispecific antibody is faricimab.

13. The method of claim 5, wherein the bispecific antibody is administered in a dose of about 6 mg.

14. The method of claim 5, wherein the bispecific antibody is administered every 12 weeks or less frequently.

15. The method of claim 5, wherein the bispecific antibody is administered every 16 weeks or less frequently.

16. The method of claim 15, wherein the bispecific antibody is administered following a treatment initiation, wherein the treatment initiation comprises 3 to 7 monthly (e.g., every 4 weeks) administrations.

17. The method of claim 5, wherein the bispecific antibody is administered at a concentration of about 120 mg/mL.

18. The method of claim 5, wherein the bispecific antibody is administered in a liquid pharmaceutical formulation comprising:
about 110 to 130 mg/mL of the bispecific antibody comprising,
about 15 to 35 mM of sodium, and
about 15 to 25 mM of a histidine acetate buffer,
and having a pH of 5.5±0.5.

19. The method of claim 18, wherein the liquid pharmaceutical formulation further comprises one or more of:
about 7.0 mM±2.0 mM methionine;
about 0.03% to 0.07% (w/v) polysorbate 20; and
about 160 mM±24 mM sucrose.

20. The method of claim 18, wherein the liquid pharmaceutical formulation has a viscosity of about 20 mPas or less, and/or a turbidity of about 30 FTU or less, and/or an ionic strength between about 20 and 50, and/or essentially free of visible particles.

21. The method of claim 5, wherein the bispecific antibody is administered intravitreally.

22. The method of claim 5, wherein the bispecific antibody is administered using a prefilled syringe.


SEQUENCE LISTING (25 sequences)

SEQ ID NO: 1 - protein, 14 aa, synthetic construct
YPYYYGTSHWYFDV

SEQ ID NO: 2 - protein, 17 aa, synthetic construct
WINTYTGEPTYAADFKR

SEQ ID NO: 3 - protein, 5 aa, synthetic construct
HYGMN

SEQ ID NO: 4 - protein, 9 aa, synthetic construct
QQYSTVPWT

SEQ ID NO: 5 - protein, 7 aa, synthetic construct
FTSSLHS

SEQ ID NO: 6 - protein, 11 aa, synthetic construct
SASQDISNYLN

SEQ ID NO: 7 - protein, 123 aa, synthetic construct [sequence omitted]

SEQ ID NO: 8 - protein, 107 aa, synthetic construct [sequence omitted]

SEQ ID NO: 9 - protein, 20 aa, synthetic construct
SPNPYYYDSSGYYYPGAFDI

SEQ ID NO: 10 - protein, 17 aa, synthetic construct
WINPNSGGTNYAQKFQG

SEQ ID NO: 11 - protein, 5 aa, synthetic construct
GYYMH

SEQ ID NO: 12 - protein, 11 aa, synthetic construct
QVWDSSSDHWV

SEQ ID NO: 13 - protein, 7 aa, synthetic construct
DDSDRPS

SEQ ID NO: 14 - protein, 11 aa, synthetic construct
GGNNIGSKSVH

SEQ ID NO: 15 - protein, 129 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 16 - protein, 110 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSS

SEQ ID NO: 17 - protein, 453 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSPGK

SEQ ID NO: 18 - protein, 463 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 19 - protein, 214 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 213 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 107 aa, Homo sapiens [sequence omitted]

SEQ ID NO: 22 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 330 aa, Homo sapiens [sequence omitted]

SEQ ID NO: 24 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 25 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF